(19) United States
(12) Patent Application Publication
(10) Pub. No.: US 2023/0151113 A1
(43) Pub. Date: May 18, 2023

(54) GPC3 CAR- T CELLS SECRETING IL-18 AND METHODS OF MAKING AND USING THE SAME

(71) Applicant: EUTILEX CO., LTD., Seoul (KR)

(72) Inventors: Byoung S. KWON, Seoul (KR); Kwanghee KIM, Seoul (KR); Jiwon CHUNG, Seoul (KR); Young Gyoon CHANG, Seoul (KR); Bo-Rim YI, Seoul (KR); Jungyun LEE, Seoul (KR); Seunghyun LEE, Seoul (KR); Sun-Woo IM, Seoul (KR); Jinkyung CHOI, Seoul (KR); Hyuntae SON, Seoul (KR); Hye Mi LEE, Seoul (KR)

(73) Assignee: EUTILEX CO., LTD., Seoul (KR)

(21) Appl. No.: 17/906,722

(22) PCT Filed: Mar. 18, 2021
(86) PCT No.: PCT/IB2021/052292

§ 371 Date: Sep. 19, 2022

Related U.S. Application Data
(60) Provisional application No. 63/043,401, filed on Jun. 24, 2020.
(60) Provisional application No. 63/004,912, filed on Apr. 3, 2020.
(60) Provisional application No. 62/991,497, filed on Mar. 18, 2020.

(51) Int. Cl. C07K 16/30; C12N 5/0783; C12N 15/86; C07K 14/54; C07K 14/55; A61K 35/17; A61P 35/00; C07K 14/705; C07K 14/725

(52) U.S. Cl. CPC C07K 16/303; C12N 5/0636; C12N 15/86; C07K 14/54; C07K 14/55; A61K 35/17; A61P 35/00; C07K 14/70578; C07K 14/7051; C12N 2830/002; C12N 2740/15043; C07K 2317/565; C07K 2319/03; C07K 2319/02; C07K 2319/33; A61K 2039/5156

(57) ABSTRACT

Provided herein are CAR-T compositions that are directed to GPC3, including a chimeric receptor, and engineered immune cells to GPC3. The disclosure also provides vectors, compositions, and methods of treatment using GPC3 antigen binding molecules and engineered immune cells, optionally in combination with expression of IL-18. GPC3 CAR compositions provided herein can be used for the treatment of certain cancers.

### CROSS-REFERENCE TO RELATED APPLICATIONS

This application claims priority to U.S. Provisional Application Ser. No. 62/991,497, filed on Mar. 18, 2020; U.S. Provisional Application Ser. No. 63/004,912, filed on Apr. 3, 2020; and U.S. Provisional Application Ser. No. 63/043,401, filed on Jun. 24, 2020, each of which is incorporated herein by reference in its entirety.

### BACKGROUND

Cancer remains one of the leading causes of death in the world. Recent statistics report that 13% of the world population dies from cancer. According to estimates from the International Agency for Research on Cancer (IARC), in 2012 there were 14.1 million new cancer cases and 8.2 million cancer deaths worldwide. By 2030, the global burden is expected to grow to 21.7 million new cancer cases and 13 million cancer deaths due to population growth and aging and exposure to risk factors such as smoking, unhealthy diet and physical inactivity. Further, pain and medical expenses for cancer treatment cause reduced quality of life for both cancer patients and their families.

T cells engineered with chimeric antigen receptors (CAR-T) have great therapeutic potential for treating diseases such as cancers. CAR-T therapeutics confer powerful target affinity and signaling function on T cell. However, the impressive efficacy of CAR-T therapies are frequently accompanied by severe side effects, such as cytokine release syndrome (CRS). Thus there remains an unmet need to develop CAR-T therapeutics and strategies that have reduced side effects.

### SUMMARY

Provided herein are immune cells comprising: a chimeric antigen receptor (CAR), wherein the CAR comprises an extracellular antigen-binding domain that binds specifically to glypican-3 (GPC3), wherein the extracellular antigen-binding domain comprises: a light chain variable domain comprising VL CDRs 1, 2, and 3 and a heavy chain variable domain comprising VH CDRs 1, 2, and 3, wherein the VL CDRs 1, 2, and 3 comprise SEQ ID NOs: 1, 2, and 3, and the VH CDRs 1, 2, and 3 comprise SEQ ID NOs: 4, 5, and 6, and a transmembrane domain, and an intracellular signaling domain; and an exogenous nucleic acid comprising a sequence encoding interleukin-18.

In some embodiments, the light chain variable domain comprises a sequence that is at least 80% identical to SEQ ID NO: 10. In some embodiments, the light chain variable domain comprises a sequence that is at least 90% identical to SEQ ID NO: 10. In some embodiments, the light chain variable domain comprises a sequence that is at least 96% identical to SEQ ID NO: 10. In some embodiments, the heavy chain variable domain comprises a sequence that is at least 80% identical to SEQ ID NO: 8. In some embodiments, the heavy chain variable domain comprises a sequence that is at least 90% identical to SEQ ID NO: 8. In some embodiments, the heavy chain variable domain comprises a sequence that is at least 96% identical to SEQ ID NO: 8. In some embodiments, the interleukin-18 is a human interleukin-18. In some embodiments, the human interleukin-18 comprises a sequence that is at least 80% identical to SEQ ID NO: 11 or 12. In some embodiments, the human interleukin-18 comprises a sequence that is at least 90% identical to SEQ ID NO: 11 or 12. In some embodiments, the human interleukin-18 comprises a sequence that is at least 96% identical to SEQ ID NO: 11 or 12.

In some embodiments, the sequence encoding interleukin-18 further includes a sequence encoding a secretion signal sequence. In some embodiments, the secretion signal sequence is an interleukin-2 secretion signal sequence. In some embodiments, the interleukin-2 secretion signal sequence comprises a sequence of SEQ ID NO: 13 or 14.

In some embodiments, the exogenous nucleic acid further comprises a promoter operably linked to the sequence encoding interleukin-18. In some embodiments, the promoter is a constitutive promoter. In some embodiments, the promoter is an inducible promoter. In some embodiments, the promoter is an NFAT promoter.

In some embodiments, the antigen-binding domain is humanized. In some embodiments, the antigen-binding domain is human. In some embodiments, the antigen-binding domain is an scFv.

In some embodiments, the transmembrane domain is a transmembrane domain selected from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6. In some embodiments, the transmembrane domain is a transmembrane domain from CD8alpha.

In some embodiments, the intracellular signaling domain comprises an intracellular signaling domain from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3epsilon, CD3gamma, CD3zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRTAM, a cytokine receptor, DAP-10, DNAM1 (CD226), Fc gamma receptor, GADS, GITR, HVEM (LIGHTR), IA4, ICAM-1, Ig alpha (CD79a), IL-2Rbeta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08, lymphocyte function-associated antigen-1 (LFA-1), a MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecules (SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6, or any combination thereof. In some embodiments, the intracellular signaling domain is from 4-1BB and CD3zeta.

In some embodiments, the immune cell is a human immune cell. In some embodiments, the human immune cell is an autologous human immune cell. In some embodiments, the human immune cell is an allogeneic human immune cell. In some embodiments, the immune cell is a T cell. In some embodiments, the immune cell is an NK cell. In some embodiments, the immune cell secretes the IL-18 encoded by the exogenous nucleic acid.

Provided herein are pharmaceutical compositions comprising any of the immune cells described herein and a pharmaceutically acceptable carrier.

Provided herein are kits comprising any of the pharmaceutical compositions described herein.

Provided herein are methods of treating a subject having a glypican-3-associated cancer, the method comprising administering to the subject any of the immune cells described herein or any of the pharmaceutical compositions described herein.

### BRIEF DESCRIPTION OF DRAWINGS

FIG. 1 shows structure of GPC3 CAR lentiviral plasmid co-expressing IL-18.

FIG. 2 shows Enzyme Mapping results after pELPS-huGC33-P2A-IL18 cloning.

FIG. 3 shows Enzyme Mapping results after pELPS-huGC33-NFAT-IL18 and pELPS-huGC33-polyA-IL18-NFAT cloning.

FIG. 4A is a graph showing cell growth by comparing total fold expansion of untreated PBMC cells, huGC33 VHVL, huGC33-VHVL-P2A-IL18, and huGC33-VHVL-NFAT-IL18 CAR-T cells.

FIG. 4B is a set of bar graphs comparing fold expansion of untreated PBMC cells, huGC33 VHVL, huGC33-VHVL-P2A-IL18, and huGC33-VHVL-NFAT-IL18 CAR-T cells in vitro on day 7, day 9, day 11, and day 14 of cell culture.

FIG. 4C is a set of bar graphs comparing cell viability of untreated PBMC cells, huGC33 VHVL, huGC33-VHVL-P2A-IL18, and huGC33-VHVL-NFAT-IL18 CAR-T cells in vitro on day 7, day 9, day 11, and day 14 of cell culture.

FIG. 4D shows results from Luciferase-based cytotoxicity assays with untreated PBMC cells, huGC33 VHVL, huGC33-VHVL-P2A-IL18 and huGC33-VHVL-NFAT-IL18 CAR-T cells in vitro, wherein the effector (E):target (T) cell ratio (E:T) can be 10:1, 3:1, 1:1, or 0.3:1. Results show that the different CAR-T cell groups showed similar in vitro killing activity.

FIG. 4E shows FACS analyses of T cells transduced with huGC33 VHVL, huGC33-VHVL-P2A-IL18 and huGC33-VHVL-NFAT-IL18 for CAR expression on day 7.

FIG. 4F shows FACS analyses of T cells transduced with huGC33 VHVL, huGC33-VHVL-P2A-IL18 and huGC33-VHVL-NFAT-IL18 for CAR expression on day 9.

FIG. 4G shows FACS analyses of T cells transduced with huGC33 VHVL, huGC33-VHVL-P2A-IL18 and huGC33-VHVL-NFAT-IL18 for CAR expression on day 11.

FIG. 4H shows FACS analyses of T cells transduced with huGC33 VHVL, huGC33-VHVL-P2A-IL18 and huGC33-VHVL-NFAT-IL18 for CAR expression on day 14.

FIG. 5A is a set of graphs showing tumor growth in animal models after huGC33-VHVL-P2A-IL18 and huGC33-VHVL-NFAT-IL18 CAR-T cell injections.

FIG. 5B is a set of graphs showing change of CAR-T cell percentage in blood of the animals after huGC33-VHVL-P2A-IL18 and huGC33-VHVL-NFAT-IL18 CAR-T cell injections. Results show that the group of mice which received injection of huGC33-VHVL-NFAT-IL18 showed the highest increase of level of CAR-T cells in the blood.

FIG. 6 shows the change of IL-18 concentration in blood of the animals over a 14-day period after injection of GPC3 VHVL NFAT IL-18 CAR-T cell injections. Upper panel shows results for each group and lower panel shows results for each animal that received an injection. Results show that the concentration of IL-18 in blood serum reached about 20 pg/mL only in the mice which received huGC33-VHVL-NFAT-IL18 CAR-T cell injections.

FIG. 7A is a graph comparing in vitro cell growth of huGC33-VHVL-NFAT-IL18 and huGC33-VHVL-IL18-NFAT CAR-T cells by measuring fold expansion for each group of cells, untreated PBMC cells, CD19 CAR-T, huGC33 VHVL, huGC33-VHVL-NFAT-IL18, and huGC33-VHVL-IL18-NFAT CAR-T cells over a 12 day period of cell culture. The group of huGC33-VHVL-NFAT-IL18 CAR-T cells yielded the largest fold expansion at 12 days of 714.0 fold. The groups of huGC33-VHVL-IL18-NFAT and huGC33-VHVL yielded similar expansion at 12 days of just under 600 fold. The group of untreated PBMC cells demonstrated a total fold expansion of approximately 490 fold. The group of CD19-CAR-T cells yielded an approximately 338 fold expansion at 12 days.

FIG. 7B is a set of bar graphs comparing fold expansion for untreated PBMC cells, CD19 CAR-T, huGC33 VHVL, huGC33-VHVL-NFAT-IL18 and huGC33-VHVL-IL18-NFAT CAR-T cells on day 5, day 7, day 9, and day 12. Results show no significant difference in cell growth of each group of CAR-T cells.

FIG. 7C is a set of bar graphs comparing cell viability of each group of untreated PBMC cells, CD19 CAR-T, huGC33 VHVL, huGC33-VHVL-NFAT-IL18 and huGC33-VHVL-IL18-NFAT CAR-T cells on day 5, day 7, day 9, and day 12. Results show no significant different in cell viability of each group of CAR-T cells.

FIG. 7D shows LDH-based cytotoxicity assay with untreated PBMC cells, CD19 CAR-T, huGC33 VHVL, huGC33-VHVL-NFAT-IL18 and huGC33-VHVL-IL18-NFAT CAR-T cells, wherein the effector (E):target (T) cell ratio (E:T) can be 10:1, 3:1, 1:1, or 0.3:1. Results show that huGC33 VHVL, huGC33 VHVL-NFAT-IL18 and huGC33-VHVL-IL18-NFAT CAR-T cells showed similar in vitro killing activity, while the untreated PBMC cells and CD19 CAR-T cells did not show significant in vitro killing activity.

FIG. 7E show results from FACS analyses of untreated PBMC cells, CD19 CAR-T, huGC33 VHVL, huGC33-VHVL-NFAT-IL18 and huGC33-VHVL-IL18-NFAT CAR-T cells on day 7.

FIG. 7F show results from FACS analyses of untreated PBMC cells, CD19 CAR-T, huGC33 VHVL, huGC33-VHVL-NFAT-IL18 and huGC33-VHVL-IL18-NFAT CAR-T cells on day 9.

FIG. 7G show results from FACS analyses of untreated PBMC cells, CD19 CAR-T, huGC33 VHVL, huGC33-VHVL-NFAT-IL18 and huGC33-VHVL-IL18-NFAT CAR-T cells on day 12.

FIG. 7H shows IL18 concentration with untreated PBMC cells, CD19 CAR-T, huGC33 VHVL, huGC33-VHVL-NFAT-IL18 and huGC33-VHVL-IL18-NFAT CAR-T cells.

FIG. 8A is a set of graphs showing tumor growth in animal models after untreated PBMC cells, CD19 CAR-T, huGC33 VHVL, huGC33-VHVL-NFAT-IL18 and huGC33-VHVL-IL18-NFAT CAR-T cell injections. Administration of huGC33-VHVL-NFAT-IL18 at 0.25 million cells suppressed tumor size in all 5 mice tested.

FIG. 8B is a set of graphs showing change of CAR-T cell percentage in blood of the animals after untreated PBMC cells, CD19 CAR-T, huGC33 VHVL, huGC33-VHVL-NFAT-IL18 and huGC33-VHVL-IL18-NFAT CAR-T cell injections. Administration of huGC33-VHVL-NFAT-IL18 0.25 million cells shows proliferation of huGC33-VHVL-NFAT-IL18 CAR-T cells during the time of tumor growth, and a decrease of huGC33-VHVL-NFAT-IL18 CAR-T cells after tumor suppression.

FIG. 9A is a set of graphs showing tumor growth in animal models after huGC33-VHVL-NFAT-IL18 and huGC33-VHVL-IL18-NFAT CAR-T cell injections. Administration of huGC33-VHVL-NFAT-IL18 0.25 million cells suppressed tumor size in all 3 mice tested.

FIG. 9B is a set of graphs showing change of CAR-T cell percentage in blood of the animals after huGC33-VHVL-NFAT-IL18 and huGC33-VHVL-IL18-NFAT CAR-T cell injections. Administration of huGC33-VHVL-NFAT-IL18 0.25 million cells shows proliferation of huGC33-VHVL-NFAT-IL18 CAR-T cells during the time of tumor growth, and a decrease of huGC33-VHVL-NFAT-IL18 CAR-T cells after tumor suppression.

FIG. 10A is a graph showing tumor growth in the animals wherein GPC3 positive cell line PLC/PRF/5-GL was used, after huGC33-VHVL-NFAT-IL18 injections. Administration of huGC33-VHVL-NFAT-IL18 1 million cells in PLC/PRF/5-GL cells suppressed tumor size at least 28 days after huGC33-VHVL-NFAT-IL18 CAR-T administration.

FIG. 10B is a graph showing tumor growth in the animals wherein GPC3 negative cell line SK-HEP-1 was used, after huGC33-VHVL-NFAT-IL18 injections. Administration of huGC33-VHVL-NFAT-IL18 1 million cells in SK-Hep-1 cells did not suppress tumor size at least 21 days after huGC33-VHVL-NFAT-IL18 CAR-T administration.

### DETAILED DESCRIPTION

This disclosure describes T cells engineered with chimeric antigen receptors (CAR-T) that include a GPC3 antigen binding domain and an IL-18 encoding sequence, as well as methods of making and using the same.

### Definitions

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies, polyclonal antibodies, and fragments thereof. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is or comprises at least a portion of a chimeric antigen receptor (CAR).

Antigen: The term “antigen”, as used herein, refers to an agent that binds to an antibody agent. In some embodiments, an antigen binds to an antibody agent and may or may not induce a particular physiological response in an organism. In general, an antigen may be or include any chemical entity such as, for example, a small molecule, a nucleic acid, a polypeptide, a carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some certain embodiments, an antigen is present in a cellular context (e.g., an antigen is expressed on the surface of a cell or expressed in a cell). In some embodiments, an antigen is a recombinant antigen.

Antigen binding domain: As used herein, refers to an antibody agent or portion thereof that specifically binds to a target moiety or entity. Typically, the interaction between an antigen binding domain and its target is non-covalent. In some embodiments, a target moiety or entity can be of any chemical class including, for example, a carbohydrate, a lipid, a nucleic acid, a metal, a polypeptide, or a small molecule. In some embodiments, an antigen binding domain may be or comprise a polypeptide (or complex thereof). In some embodiments, an antigen binding domain is part of a fusion polypeptide. In some embodiments, an antigen binding domain is part of a chimeric antigen receptor (CAR).

Associated with: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

CDR: as used herein, refers to a complementarity determining region within a variable region of an antibody agent. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

Chemotherapeutic Agent: The term “chemotherapeutic agent”, has used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine.

Engineered: In general, the term “engineered” refers to the aspect of having been manipulated by the hand of man. For example, a polypeptide is considered to be “engineered” when the polypeptide sequence manipulated by the hand of man. For example, in some embodiments of the present invention, an engineered polypeptide comprises a sequence that includes one or more amino acid mutations, deletions and/or insertions that have been introduced by the hand of man into a reference polypeptide sequence. In some embodiments, an engineered polypeptide includes a polypeptide that has been fused (i.e., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Operably linked: as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Pharmaceutical composition: As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibody agents, antibody fragments, biologically active portions thereof, and/or characteristic portions thereof.

Prevent or prevention: as used herein when used in connection with the occurrence of a disease, disorder, and/or condition, refers to reducing the risk of developing the disease, disorder and/or condition and/or to delaying onset and/or severity of one or more characteristics or symptoms of the disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically Effective Amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in any of the pro-angiogenic markers discussed above, an increase in anti-angiogenic markers described herein, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence. In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual 2^{nd} ed., Cold Spring Harbor Laboratory Press, Cold Spring Harbor, N.Y. (1989)), which is incorporated herein by reference for any purpose.

### Engineered Immune Cells

As used herein, “immune cells” refer to cells of the immune system which can be categorized as lymphocytes (e.g., T cells, B cells, and NK cells), neutrophils, and monocytes/macrophages. In some embodiments, the immune cell is a T cell. In some embodiments, the immune cell is an NK cell. In some embodiments, an immune cell is an engineered immune cell, which means the immune cell has been genetically modified to express a non-naturally occurring protein (e.g., a chimeric antigen receptor) or to include an exogenous nucleic acid.

The immune cells (e.g., T cells) may be modified in one or more than one manner. Immune cells (e.g., T cells) may express at least one non-natural molecule that is a receptor for an antigen that is present on the surface of one or more types of cells. In some embodiments, immune cells, include immune cells (e.g., T cells) that are not found in nature because they are engineered to comprise or express at least one synthetic molecule that is not found in nature. In specific embodiments, the immune cells (e.g., T cells) are engineered to express at least one chimeric antigen receptor (CAR), including a CAR that targets a specific tumor antigen, such as glypican-3 (GPC3). In specific embodiments, the immune cell can be a T cell, e.g., a CD4+ T cell, a CD8+ T cell, a Treg cell, a Th1 T cell, a Th2 T cell, a Th17 T cell, an unspecific T cell, or a population of T cells that comprises a combination of any of the foregoing. Immune cells (e.g., T cells) engineered with chimeric antigen receptors (CAR T cells) have great therapeutic potential for treating cancers. With a CAR, a receptor can be programmed to recognize an antigen, which when bound, activate immune cells to kill the cell expressing that antigen. Therefore, immune cells expressing CAR(s) for an antigen expressed on a tumor cell can target and kill the tumor cell. For example, recent clinical trials of a CD19-targeted CAR-transduced T cell (CD19-CAR T cell) against hematologic malignancies showed a strong effect of CAR T technology. (Kochenderfer, J. N. et al. (2010) Blood 116: 4099-4102; Porter, D. L., et al. (2011) N. Engl. J. Med. 365: 725-733; Grupp, S. A. et al. (2013) N. Engl. J. Med. 368: 1509-1518; Kochenderfer, J. N. et al. (2015) J. Clin. Oncol. 33: 540-549; Brown, C. E. et al. (2016) N. Engl. J. Med. 375: 2561-2569). The clinical success of CAR T is attributed, at least in part, to the fusion structure of the CAR, which is made by artificially combining a high-affinity antigen-binding domain with multiple signaling domains (Maus, M. V. et al. (2014) Blood 123: 2625-2635; van der Stegen, S. J. et al. (2015) Nat. Rev. Drug Discov. 14: 499-509).

CARs comprise an extracellular antigen-binding domain, a transmembrane domain and an intracellular signaling domain. In some embodiments, the extracellular antigen-binding domain comprises a single chain variable fragment (scFv) that is capable of recognizing a tumor-associated antigen, the transmembrane domain employs the transmembrane domain from molecules such as CD8 and CD28, and the intracellular signaling domain employs an immunoreceptor tyrosine-based activation motif (e.g., CD3ζ) and the intracellular signaling domain of co-stimulatory signaling molecule (e.g., CD28, CD137, and CD137 (4-1BB)).

As used herein, “single chain variable fragment, scFv” refers to a fragment of antibody defined as a recombinant protein comprising a heavy chain variable domain (VH) and a light chain variable domain (VL) connected by a linker, which brings the two domains together into association such that an antigen-binding site is formed.

In some embodiments, the transmembrane domain is a transmembrane domain from a protein selected from 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM11, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6.

In some embodiments, the intracellular signaling domain comprises an intracellular signaling domain from a protein selected from 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3epsilon, CD3gamma, CD3zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRTAM, a cytokine receptor, DAP-10, DNAM1 (CD226), Fc gamma receptor, GADS, GITR, HVEM (LIGHTR), IA4, ICAM-1, Ig alpha (CD79a), IL-2Rbeta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08, lymphocyte function-associated antigen-1 (LFA-1), a MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecules (SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6, or any combination thereof. In some embodiments, the intracellular signaling domain of the chimeric receptors described herein include a 4-1BB signaling domain followed by a five amino acid sequence, which may be further combined with any other desired extracellular, transmembrane, and/or intracellular domain(s) useful in the context of the chimeric receptor. In some embodiments, the 4-1BB signaling domain followed by a five amino acid sequence is referred to as euBBz. In some embodiments, the chimeric antigen receptor further comprises an additional antigen-binding domain. In some embodiments, the additional antigen-binding domain is an scFv.

The immune cells, (e.g., T cells) can come from any source known in the art. For example, immune (e.g., T) cells can be differentiated in vitro from a hematopoietic stem cell population, or immune (e.g., T) cells can be obtained from a subject. T cells can be obtained from peripheral blood mononuclear cells (PBMCs), bone marrow, lymph node tissue, cord blood, thymus tissue, tissue from a site of infection, ascites, pleural effusion, spleen tissue, or tumors. In addition, immune (e.g., T) cells can be derived from one or more immune cell lines available in the art. In some embodiments, T cells can be obtained from blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety. Other non-limiting examples can be found in International Application No. PCT/US2015/014520 (published as WO2015/120096) and in International Application No. PCT/US2016/057983 (published as WO2017/070395), each of which is herein incorporated by reference in its entirety.

In some embodiments, the immune cells are autologous T cells. In some embodiments, the immune cells are obtained from a subject that is not the patient. In some embodiments, T cells for using in a therapeutic method are syngeneic (the donor and the recipients are different but are identical twins). In some embodiments, T cells for using in a therapeutic method are allogenic (from the same species but different donor) as the recipient subject. In some embodiments, the T cells are autologous stem cells (for autologous stem cell therapy or ASCT). In some embodiments, the immune cells are non-autologous T-cells. In some embodiments, the immune cells are obtained from a healthy donor. In some embodiments, the immune cells are obtained from a patient afflicted with a cancer or a tumor.

T cells can be engineered to express, for example, chimeric antigen receptors (CARs). In some embodiments, CAR-T cells can be engineered to express an extracellular single chain variable fragment (scFv). In some embodiments, the CAR is engineered such that the costimulatory domain is expressed as a separate polypeptide chain. Exemplary CAR-T cell therapies and constructs are described in U.S. Patent Publication Nos. 2013/0287748, 2014/0227237, 2014/0099309, and 2014/0050708, each of which are herein incorporated by reference in their entirety.

### GPC3

Glypican-3 (GPC3) is a cell surface protein encoded by the GPC3 gene in humans and an oncofetal antigen re-expressed in a high frequency of neoplastic hepatocytes. GPC3 is highly expressed in fetal liver and not expressed in normal adult liver tissue, but its expression is reactivated in hepatocellular carcinoma, and has close association with the development of liver cancer, where the detection rate of GPC3 expression is relatively high during early stage of liver cancer and increases along with the development of liver cancer. Further, GPC3 is also expressed in tumors such as melanoma, ovarian clear cell carcinoma, yolk sac tumor, neuroblastoma and other tumors. Considering its specifically high expression in hepatocellular carcinoma, melanoma and other tumors, GPC3 has emerged as a useful immunohistochemical diagnostic test and potential biomarker.

GPC3 is a member of the proteoglycan family that functions as extracellular matrix in cell adhesion in organogenesis or as a receptor of a cell growth factor. The protein core of GPC3 comprises two subunits, and N-terminal subunit and a C-terminal subunit. A glycosyl phosphatidylinositol (GPI) anchor is added to serine at position 560 located on the carboxyl (C)-terminal side of GPC3. The GPI anchor plays a role in localizing GPC3 on cell surface through covalent binding to cell membrane lipid. Also, serine at position 495 and serine at position 509 of GPC3 are modified with a heparan sulfate chain (HS chain) wherein the HS chain is known to regulate a plurality of growth signal transduction pathways such as Wnt signal, FGF signal, and BMP signal transduction pathways. A growth signal transduction pathway involved is known to differ among the types of cancers. For example, in hepatocellular carcinoma (HCC), cells grow by the stimulation of the Wnt signal pathway.

### GPC3 CAR

The present disclosure provides, at least in part, GPC3 CAR polypeptides. As used herein, “chimeric antigen receptor (CAR)” refers to a receptor not present in nature and is capable of providing an immune effector cell with a specificity to a particular antigen. Normally, the CAR refers to a receptor used for delivering the specificity of a monoclonal antibody agent to a T cell. Generally, a CAR comprises an extracellular binding domain (Ectodomain), a transmembrane domain, and an intracellular signaling domain (Endodomain). In some embodiments, an extracellular binding domain of a CAR comprises an antigen binding domain. In some embodiments, an antigen binding domain is or comprises an antibody agent. In some embodiments, an antigen binding domain is or comprises an antibody agent that specifically binds to GPC3.

In some embodiments, the chimeric antigen receptor (CAR) polypeptide includes: i) an extracellular antigen-binding domain comprising a light chain variable domain comprising a light chain CDR1 comprising SEQ ID NO: 1; a light chain CDR2 comprising SEQ ID NO: 2; and a light chain CDR3 comprising SEQ ID NO: 3; and a heavy chain variable domain comprising a heavy chain CDR1 comprising SEQ ID NO: 4; a heavy chain CDR2 comprising SEQ ID NO: 5; and a heavy chain CDR3 comprising SEQ ID NO: 6; ii) a transmembrane domain; and iii) an intracellular signaling domain, which leads to T cell activation when an antigen binds to the antibody agent.

TABLE 1

| | SEQUENCE | SEQ ID NO: |
|---|---|---|
| Light chain CDR1 | RSSQSLVHS | 1 |
| | NGNTYLH | |
| Light chain CDR2 | KVSNRFS | 2 |
| Light chain CDR3 | SQNTHVPPT | 3 |
| Heavy chain CDR1 | DYEMH | 4 |
| Heavy chain CDR2 | ALDPKTGDT | 5 |
| | AYSQKFKG | |
| Heavy chain CDR3 | FYSYTY | 6 |

In some embodiments, the CAR polypeptide includes: i) an extracellular antigen-binding domain comprising a light chain variable domain comprising a sequence that is at least 80%, 85%, 90%, 91%, 92%, 93%, 94%, 95%, 96%, 97%, 98%, or 99% identical to SEQ ID NO: 10 and a heavy chain variable domain comprising a sequence that is at least 80%, 85%, 90%, 91%, 92%, 93%, 94%, 95%, 96%, 97%, 98%, or 99% identical to SEQ ID NO: 8; ii) a transmembrane domain; and iii) an intracellular signaling domain, which leads to T cell activation when an antigen binds to the antibody agent.

In some embodiments, the CAR polypeptide includes: i) an extracellular antigen-binding domain comprising a light chain variable domain comprising SEQ ID NO: 10 and a heavy chain variable domain comprising SEQ ID NO: 8; ii) a transmembrane domain; and iii) an intracellular signaling domain, which leads to T cell activation when an antigen binds to the antibody agent.

TABLE 2

| SEQ ID | | | |
|---|---|---|---|
| NO: | NAME | TYPE | SEQUENCE |
| 7 | huGC33 VH | Nucleotide | CAAGTGCAACTCGTACAATCAGGTGCTGAAGTCA |
| | | | AAAAGCCGGGAGCCTCTGTTAAAGTGTCCTGTAA |
| | | | AGCCAGCGGCTACACCTTTACCGATTATGAGATG |
| | | | CACTGGGTTCGGCAGGCTCCGGGCCAAGGTCTCG |
| | | | AGTGGATCGGGGCTCTTGACCCAAAGACGGGCGA |
| | | | CACGGCTTATTCACAAAAATTCAAAGGTAGGGCT |
| | | | ACTCTGACTGCCGATAAGTCCACCAGCACCGCGT |
| | | | ATATGGAGCTCTCTAGCTTGCGAAGCGAGGACAC |
| | | | GGCGGTGTACTATTGCACACGCTTCTATAGTTAC |
| | | | ACATATTGGGGTCAAGGCACGCTTGTGACCGTGT |
| | | | CTAGC |
| 8 | | Amino acid | QVQLVQSGAEVKKPGASVKVSCKASGYTFTDYEM |
| | | | HWVRQAPGQGLEWIGALDPKTGDTAYSQKFKGRA |
| | | | TLTADKSTSTAYMELSSLRSEDTAVYYCTRFYSY |
| | | | TYWGQGTLVTVSS |
| 9 | huGC33 VL | Nucleotide | GACGTCGTTATGACACAGAGTCCCCTCTCCTTGC |
| | | | CGGTGACCCTGGGTCAGCCTGCGTCCATCTCTTG |
| | | | CAGATCCTCCCAGTCTCTGGTACACTCCAACGGC |
| | | | AACACATACTTGCACTGGTACCAACAAAGACCTG |
| | | | GTCAGTCACCGCGACTTCTCATATATAAAGTTTC |
| | | | CAATAGGTTCAGTGGAGTGCCAGACAGGTTCAGT |
| | | | GGTTCAGGATCAGGCACTGATTTCACGCTTAAAA |
| | | | TCAGTCGGGTTGAGGCGGAGGACGTAGGAGTTTA |
| | | | CTATTGCAGCCAGAATACGCACGTGCCGCCTACT |
| | | | TTTGGCTCTGGAACCAAGTTGGAAATAAAG |
| 10 | | Amino acid | DVVMTQSPLSLPVTLGQPASISCRSSQSLVHSNG |
| | | | NTYLHWYQQRPGQSPRLLIYKVSNRFSGVPDRFS |
| | | | GSGSGTDFTLKISRVEAEDVGVYYCSQNTHVPPT |
| | | | FGSGTKLEIK |

### IL-18

Interleukin-18 (IL-18) is characterized as an inducer of interferon-γ (IFN-γ) expression in T cells and has been shown to activate lymphocytes and monocytes without eliciting severe dose-limiting toxicity in clinical trials. IL-18 is a proinflammatory cytokine encoded by the IL18 gene in humans. Further, it is known that IL-18 has various functions in addition to the ability to induce interferon-γ. IL-18 has functions such as activation of NF-κB, expression of Fas ligand, induction of both CC and CXC chemokines, and increase in the production of competent human immunodeficiency virus. Since IL-18 has the ability to induce interferon-γ production in T cells and macrophages, it plays an important role in Th1-type immune response and participates both in congenital immunity and in acquired immunity. IL-18 is related to the IL-1 family both in terms of structure and of function.

In some embodiments, IL-18 secreting CAR-T cells exhibit enhanced CAR-T cell in vivo expansion and significantly increased long-term survival in mouse models of hematological and solid malignancies. In some embodiments, IL-18 secreting CAR-T cells can enhance an effective endogenous anti-tumor immune response. In some embodiments, a GPC3 CAR-T cell can co-express IL-18, thereby enhancing the expansion of the CAR-T cells and increasing effectiveness of the anti-tumor immune response.

human interleukin-18
SEQ ID NO: 11
TACTTTGGCAAGCTTGAATCTAAATTATCAGTCATAAGAAATTTGAATGA
CCAAGTTCTCTTCATTGACCAAGGAAATCGGCCTCTATTTGAAGATATGA
CTGATTCTGACTGTAGAGATAATGCACCCCGGACCATATTTATTATAAGT
ATGTATAAAGATAGCCAGCCTAGAGGTATGGCTGTAACTATCTCTGTGAA
GTGTGAGAAAATTTCAACTCTCTCCTGTGAGAACAAAATTATTTCCTTTA
AGGAAATGAATCCTCCTGATAACATCAAGGATACAAAAAGTGACATCATA
TTCTTTCAGAGAAGTGTCCCAGGACATGATAATAAGATGCAATTTGAATC
TTCATCATACGAAGGATACTTTCTAGCTTGTGAAAAAGAGAGAGACCTTT
TTAAACTCATTTTGAAAAAAGAGGATGAATTGGGGGATAGATCTATAATG
TTCACTGTTCAAAACGAAGACTAG
human interleukin-18
(reverse complement)
SEQ ID NO: 12
CTAGTCTTCGTTTTGAACAGTGAACATTATAGATCTATCCCCCAATTCAT
CCTCTTTTTTCAAAATGAGTTTAAAAAGGTCTCTCTCTTTTTCACAAGCT
AGAAAGTATCCTTCGTATGATGAAGATTCAAATTGCATCTTATTATCATG
TCCTGGGACACTTCTCTGAAAGAATATGATGTCACTTTTTGTATCCTTGA
TGTTATCAGGAGGATTCATTTCCTTAAAGGAAATAATTTTGTTCTCACAG
GAGAGAGTTGAAATTTTCTCACACTTCACAGAGATAGTTACAGCCATACC
TCTAGGCTGGCTATCTTTATACATACTTATAATAAATATGGTCCGGGGTG
CATTATCTCTACAGTCAGAATCAGTCATATCTTCAAATAGAGGCCGATTT
CCTTGGTCAATGAAGAGAACTTGGTCATTCAAATTTCTTATGACTGATAA
TTTAGATTCAAGCTTGCCAAAGTA
interleukin-2 secretion
signal sequence
SEQ ID NO: 13
ATGTACAGGATGCAACTCCTGTCTTGCATTGCACTAAGTCTTGCACTTGT
CACAAACAGT
interleukin-2 secretion signal
sequence (reverse complement)
SEQ ID NO: 14
ACTGTTTGTGACAAGTGCAAGACTTAGTGCAATGCAAGACAGGAGTTGCA
TCCTGTACAT

### Nucleic Acids

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs) and/or deoxyribonucleic acids (DNAs). Provided herein are nucleic acids comprising a nucleotide sequence encoding GPC3 CARs, wherein the GPC3 CAR comprises: an extracellular antigen-binding domain that binds specifically to glypican-3 (GPC3), a transmembrane domain, and an intracellular signaling domain.

In some embodiments, nucleic acid constructs include regions that encode a GPC3 CAR. In some embodiments, nucleic acid constructs may be inserted into an expression vector or viral vector by methods known to the art, and nucleic acid molecules may be operably linked to an expression control sequence. Non-limiting examples of expression vectors include plasmid vectors, transposon vectors, cosmid vectors, and viral vectors (e.g., any adenoviral vectors (AV), cytomegaloviral (CMV) vectors, simian viral (SV40) vectors, adeno-associated virus (AAV) vectors, lentiviral vectors, and retroviral vectors). In some embodiments, the expression vector is a viral vector. In some embodiments, the viral vector is a lentiviral vector.

In some embodiments, nucleic acid constructs include regions that encode a GPC3 CAR and a sequence encoding an interleukin-18. In some embodiments, the GPC3 CAR polypeptide includes: i) an extracellular antigen-binding domain comprising a light chain variable domain encoded by a nucleic acid comprising a sequence that is at least 80%, 85%, 90%, 91%, 92%, 93%, 94%, 95%, 96%, 97%, 98%, or 99% identical to SEQ ID NO: 9 and a heavy chain variable domain encoded by a nucleic acid comprising a sequence that is at least 80%, 85%, 90%, 91%, 92%, 93%, 94%, 95%, 96%, 97%, 98%, or 99% identical to SEQ ID NO: 7; ii) a transmembrane domain; and iii) an intracellular signaling domain, which leads to T cell activation when an antigen binds to the antibody agent.

In some embodiments, the CAR polypeptide includes: i) an extracellular antigen-binding domain comprising a light chain variable domain encoded by a nucleic acid comprising SEQ ID NO: 9 and a heavy chain variable domain encoded by a nucleic acid comprising SEQ ID NO: 7; ii) a transmembrane domain; and iii) an intracellular signaling domain, which leads to T cell activation when an antigen binds to the antibody agent.

In some embodiments, the IL-18 is a human interleukin-18. In some embodiments, the IL-18 is a humanized IL-18. In some embodiments, the IL-18 is encoded by a nucleic acid comprising a sequence that is at least 80%, 85%, 90%, 91%, 92%, 93%, 94%, 95%, 96%, 97%, 98%, or 99% identical to SEQ ID NO: 11 or 12. In some embodiments, the IL-18 is encoded by a nucleic acid comprising SEQ ID NO: 11 or 12.

In some embodiments, the sequence encoding IL-18 further comprises a sequence encoding a secretion signal sequence. As used herein, “secretion signal sequence” refers to a short peptide (e.g., 16-30 amino acids) present at the N-terminus of the majority of newly synthesized proteins that are destined towards the secretory pathway. These proteins include those that reside either inside cell organelles (e.g., endoplasmic reticulum, Golgi, or endosomes), secreted from the cell, or inserted into most cellular membranes. In some embodiments, the secretion signal sequence is an interleukin-2 secretion signal sequence. In some embodiments, the interleukin-2 secretion signal sequence comprises a sequence that is at least 80%, 85%, 90%, 91%, 92%, 93%, 94%, 95%, 96%, 97%, 98%, or 99% identical to SEQ ID NO: 13 or 14. In some embodiments, the IL-2 secretion signal sequence comprises a sequence of SEQ ID NO: 13 or 14.

In some embodiments, nucleic acid constructs further comprise a promoter operably linked to the sequence encoding IL-18, wherein the promoter is disposed between the sequence encoding the CAR and the sequence encoding interleukin-18. As used herein, “promoter” refers to a region of DNA that leads to initiation of transcription of a particular gene. Promoters are located near the transcription start site of genes, upstream on the DNA (e.g., towards the 5′ region of the sense strand). In some embodiments, promoters are about 100-1000 base pairs long. In some embodiments, the promoter is a constitutive promoter. In some embodiments, the promoter is an inducible promoter. In some embodiments, the promoter is an NFAT promoter.

In some embodiments, nucleic acid constructs further comprise a sequence encoding a self-cleaving protein, wherein the sequence is disposed between the sequence encoding the CAR and the sequence encoding IL-18. In some embodiments, the self-cleaving protein sequence is a P2 self-cleaving protein sequence.

A lentiviral vector is derived from a lentivirus. Lentiviral vectors are based on the single-stranded RNA lentiviruses, which are a subclass of retrovirus. They combine the advantages of midrange cloning capacity with stable gene expression, wherein they are able to transduce dividing and non-dividing cells, including neurons. Upon infection, the lentiviral genome integrates transgenes into the host genome and promotes long-term gene expression. Lentiviral vectors, such as HIV-based vectors, are exemplary of retroviral vectors used for gene delivery. Unlike other retroviruses, HIV-based vectors are known to incorporate their passenger genes into non-dividing cells and, therefore, can be of use in treating persistent forms of disease.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers are well known in the art.

In some embodiments, nucleic acid molecules are inserted into a vector that is able to express a GPC3 CAR of the present disclosure when introduced into an appropriate cell. In some embodiments, an appropriate cell is a T cell.

### Production of GPC3 CAR-T Cells Expressing IL-18

Provided herein are methods for producing immune cells comprising a GPC3 CAR expressing IL-18. In some embodiments, the immune cell where a CAR is introduced therein is a human immune cell. In some embodiments, the immune cell is an autologous human immune cell. In some embodiments, the immune cell is an allogeneic human immune cell. In some embodiments, the immune cell is a CD4⁺ T cell (helper T cell, TH cell), a CD8⁺ T cell (cytotoxic T cell, CTL), a memory T cell, a regulatory T cell (Treg cell), an apoptotic T cell, but is not limited thereto. In some embodiments, the immune cell is an NK cell.

In some embodiments, the present disclosure provides methods of producing an engineered immune cell, comprising: introducing into an immune cell (i) a nucleic acid sequence encoding a GPC3 CAR, comprising a GPC3 antigen binding domain, and a sequence encoding an interleukin-18 (IL-18) or (ii) a vector comprising the nucleic acid encoding a GPC3 CAR, comprising a GPC3 antigen binding domain, and the nucleic acid encoding an IL-18. In some embodiments, a method of producing an engineered immune cell of the present disclosure further comprises culturing the engineered immune cell in vitro for at least 2 days, 5 days, 7 days, 8 days, 9 days, 10 days, 11 days, 12 days, 13 days, or 14 days.

In some embodiments, the present disclosure provides methods of preparing an autologous engineered immune cell of the present disclosure, comprising: providing or obtaining an analysis of binding of a GPC3 antigen binding domain to a T cell from a subject; and if the binding is less than a threshold value, engineering an immune cell from the subject to express a CAR comprising the GPC3 antigen binding domain, wherein the CAR further expresses IL-18. In some embodiments, a method of producing an autologous engineered immune cell of the present disclosure (e.g., GPC3 CAR-T cells expressing IL-18) further comprises culturing the autologous engineered immune cell in vitro for at least 2 days, 5 days, 7 days, 8 days, 9 days, 10 days, 11 days, 12 days, 13 days, or 14 days.

Any method known in the art for expressing a CAR in immune cells can be used in the context of the present disclosure. For example, there are various nucleic acid vectors for expression known in the art, such as linear polynucleotides, polynucleotides to which an ionic or amphiphilic compound is bound, plasmids, or viral vectors, though the present disclosure is not limited thereto. In some embodiments, a vector for expression of a CAR in immune cells may be or include an autonomously replicating plasmid or virus or derivative thereof. Viral vectors can include, but are not limited to adenoviral vector, adeno-associated viral vector, retroviral vector, etc. In some embodiments a lentiviral vector, which is a retroviral vector, can be used. In some embodiments, a vector is a non-plasmid and a non-viral compound, such as, for example, a liposome.

The present disclosure encompasses the recognition that GPC3 CAR-T cells co-expressing IL-18, generated by the methods described herein may be therapeutically useful (e.g., for the treatment of cancer).

### Therapeutic Applications

Provided herein are methods of treating a subject having a glypican-3-associated cancer, wherein the method comprises administering to a subject a composition that comprises or delivers an immune cell comprising a GPC3 CAR expressing IL-18.

A “glypican-3-associated cancer” is a cancer that is characterized by a cancer cell having glypican-3 present on its surface. GPC3, a membrane-bound heparan sulfate proteoglycan, is overexpressed in approximately 70% to 80% of hepatocellular carcinomas, but is not expressed commonly in healthy tissues. In addition, GPC3 overexpression is found in several tumors, most notably in hepatocellular carcinomas, hepatoblastoma, germ cell tumors (e.g., yolk sac tumors, choriocarcionomas), Wilms tumor, gastric carcinoma, non-small lung cancer, and thyroid cancer.

Interleukin-18 (IL-18) is a cytokine that enhances innate and adaptive immune responses, and is produced by activated immune cells, such as monocytes, macrophages, dendritic cells (DCs), neutrophils, natural killer (NK) cells, T cells, and B cells. Various types of cancer produce IL-18, and IL-18 induces cell migration, invasion, and proliferation, resulting in increased metastasis and tumor growth. IL-18 transcription level is known to be increased in most cancer types, including but not limited to, cervical squamous cell carcinoma and endocervical adenocarcinoma, colon adenocarcinoma, glioblastoma, kidney renal papillary cell carcinoma, acute myeloid leukemia, pancreatic adenocarcinoma, breast cancer, brain cancer, and pancreatic cancer.

Cancer can refer to a broad group of diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. Cancer or cancer tissue may include a tumor.

Cancers suitable for treatment by a method of the present disclosure can include, but are not limited to, bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, and prostate cancer. In some embodiments, a cancer for treatment by a method of the present disclosure can include may include, but is not limited to, carcinoma, lymphoma (e.g., Hodgkin's and non-Hodgkin's lymphomas), blastoma, sarcoma and leukemia. In some embodiments, cancer may include squamous cell carcinoma, small cell lung cancer, non-small cell lung cancer, lung adenocarcinoma, squamous cell carcinoma of the lung, peritoneal cancer, hepatocellular carcinoma, gastric cancer, pancreatic cancer, glioma, cervical cancer, ovarian cancer, liver cancer, bladder cancer, hepatocellular carcinoma, breast cancer, colon cancer, colorectal cancer, endometrial or uterine carcinoma, salivary carcinoma, kidney cancer, prostate cancer, vulvar cancer, thyroid cancer, liver carcinoma, leukemia and other lymphoproliferative disorders, and various types of head and neck cancer. In some embodiments, the cancer can be an embryonal tumor (Wilms tumor, hepatoblastoma, rhabdoid, neuroblasoma), germ cell tumor (yolk sac tumor, immature teratoma, and embryonal carcinoma), carcinoma (hepatocellular carcinoma and pulmonary squamous cell carcinoma), sarcoma (malignant rhabdoid tumor and RMS), or malignant melanoma. In some embodiments, a glypican-3-associated cancer is a liver cancer.

The immune cells (e.g., GPC3 CAR-T cells expressing IL-18) may be administered at a therapeutically effective amount to a patient in need thereof. For example, a therapeutically effective amount of the immune cells (e.g. GPC3 CAR-T cells expressing IL-18) may be at least about 10⁴ cells, at least about 10⁵ cells, at least about 10⁶ cells, at least about 10⁷ cells, at least about 10⁸ cells, at least about 10⁹, or at least about 10¹⁰. In some embodiments, a therapeutically effective amount of T cells is about 10⁴ cells, about 10⁵ cells, about 10⁶ cells, about 10⁷ cells, about 10⁸ cells, about 10⁹ cells, or about 10¹⁰ cells. In some embodiments, the therapeutically effective amount of the T cells is between about 0.1×10⁶ and about 2×10¹⁰ T cells (e.g., about 0.1×10⁶ and about 2×10¹⁰ T cells, about 0.2×10⁶ and about 2×10¹⁰ T cells, about 0.4×10⁶ and about 2×10¹⁰ T cells, about 0.6×10⁶ and about 2×10¹⁰ T cells, about 0.8×10⁶ and about 2×10¹⁰ T cells, about 1.0×10⁶ and about 2×10¹⁰ T cells, about 2.0×10⁶ and about 2×10¹⁰ T cells, about 3.0×10⁶ and about 2×10¹⁰ T cells, about 4.0×10⁶ and about 2×10¹⁰ T cells, about 5.0×10⁶ and about 2×10¹⁰ T cells, about 6.0×10⁶ and about 2×10¹⁰ T cells, about 7.0×10⁶ and about 2×10¹⁰ T cells, about 8.0×10⁶ and about 2×10¹⁰ T cells, about 9.0×10⁶ and about 2×10¹⁰ T cells, about 1.0×10⁷ and about 2×10¹⁰ T cells, about 2.0×10⁷ and about 2×10¹⁰ T cells, about 3.0×10⁷ and about 2×10¹⁰ T cells, about 4.0×10⁷ and about 2×10¹⁰ T cells, about 5.0×10⁷ and about 2×10¹⁰ T cells, about 6.0×10⁷ and about 2×10¹⁰ T cells, about 7.0×10⁷ and about 2×10¹⁰ T cells, about 8.0×10⁷ and about 2×10¹⁰ T cells, about 9.0×10⁷ and about 2×10¹⁰ T cells, about 1.0×10⁸ and about 2×10¹⁰ T cells, about 2.0×10⁸ and about 2×10¹⁰ T cells, about 3.0×10⁸ and about 2×10¹⁰ T cells, about 4.0×10⁸ and about 2×10¹⁰ T cells, about 5.0×10⁸ and about 2×10¹⁰ T cells, about 6.0×10⁸ and about 2×10¹⁰ T cells, about 7.0×10⁸ and about 2×10¹⁰ T cells, about 8.0×10⁸ and about 2×10¹⁰ T cells, about 9.0×10⁸ and about 2×10¹⁰ T cells, about 1.0×10⁹ and about 2×10¹⁰ T cells, about 2.0×10⁹ and about 2×10¹⁰ T cells, about 3.0×10⁹ and about 2×10¹⁰ T cells, about 4.0×10⁹ and about 2×10¹⁰ T cells, about 5.0×10⁹ and about 2×10¹⁰ T cells, about 6.0×10⁹ and about 2×10¹⁰ T cells, about 7.0×10⁹ and about 2×10¹⁰ T cells, about 8.0×10⁹ and about 2×10¹⁰ T cells, about 9.0×10⁹ and about 2×10¹⁰ T cells, or about 1.0×10¹⁰ and about 2×10¹⁰ T cells. In some embodiments, the therapeutically effective amount of the T cells is about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, about 1.5×10⁸, about 1.6×10⁸, about 1.7×10⁸, about 1.8×10⁸, about 1.9×10⁸, or about 2.0×10⁸ T cells.

In some embodiments, a therapeutically effective amount of the GPC3 CAR T cells expressing IL-18 is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, a therapeutically effective amount of immune cells (e.g., GPC3 CAR-T cells expressing IL-18) is between about 1×10⁶ and about 2×10⁶ T cells per kg body weight up to a maximum dose of about 1×10¹⁰ T cells. In some embodiments, the therapeutically effective amount of the T cells is about 1×10⁶ or about 2×10⁶ T cells per kg body weight up to a maximum dose of about 1×10¹⁰ T cells.

The number of cells will depend upon the ultimate use for which the composition is intended as will the type of cells included therein. For example, in some embodiments, a population of T cells comprising a GPC3 CAR expressing IL-18 will contain greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, or greater than 90% of such cells. In some embodiments, a population of T cells comprising a GPC3 CAR expressing IL-18 will contain about 10% to about 90%, about 10% to about 80%, about 10% to about 70%, about 10% to 60%, about 10% to about 50%, about 10% to about 40%, about 10% to about 30%, about 10% to about 20%, about 10% to about 15%, about 15% to about 90%, about 15% to about 80%, about 15% to about 70%, about 15% to about 60%, about 15% to about 50%, about 15% to about 40%, about 15% to about 30%, about 15% to about 20%, about 20% to about 90%, about 20% to about 80%, about 20% to about 70%, about 20% to about 60%, about 20% to about 50%, about 20% to about 40%, about 20% to about 30%, about 30% to about 90%, about 30% to about 80%, about 30% to about 70%, about 30% to about 60%, about 30% to about 50%, about 30% to about 40%, about 40% to about 90%, about 40% to about 80%, about 40% to about 70%, about 40% to about 60%, about 40% to about 50%, about 50% to about 90%, about 50% to about 80%, about 50% to about 70%, about 50% to about 60%, about 60% to about 90%, about 60% to about 80%, about 60% to about 70%, about 70% to about 90%, about 70% to about 80%, or about 80% to about 90% of such T cells. In some embodiments, a population of T cells for administration is in a volume of a liter or less. In some embodiments, T cells for administration are in a volume of less than 500 ml, less than 250 ml, or 100 ml or less. In some embodiments, a density of the desired T cells is typically greater than 10⁶ cells/ml and generally is greater than 10⁷ cells/ml, generally 10 cells/ml or greater. A clinically relevant number of immune cells can be apportioned into multiple infusions that cumulatively equal or exceed 10⁷ cells, 10⁸ cells, 10⁹ cells, 10¹⁰ cells, 10¹¹ cells, or 10¹² cells.

In some embodiments, a composition may be administered to a patient parenterally. In some embodiments, a composition that comprises or delivers a T cell comprising a GPC3 CAR expressing IL-18 may be parenterally administered to a patient in one or multiple administrations. In some embodiments, a composition that comprises or delivers a T cell comprising a GPC3 CAR expressing IL-18 may be parenterally administered to a patient once every day, once every 2 to 7 days, once every week, once every two weeks, once every month, once every three months, or once every 6 months.

In some embodiments, the present disclosure provides methods of inducing an immune response in a subject in need thereof, the method comprising administering to the subject a composition that comprises or delivers a T cell comprising a GPC3 CAR expressing IL-18. In some embodiments a T cell comprising a GPC3 CAR expressing IL-18 is an autologous T cell. In some embodiments, the present disclosure provides methods of inducing an immune response in a subject in need thereof, the method comprising administering to the subject a composition that comprises or delivers a T cell comprising a nucleic acid and/or vector encoding a GPC3 CAR expressing IL-18. In some embodiments a T cell comprising a nucleic acid and/or vector encoding a GPC3 CAR expressing IL-18 is an autologous T cell. In some embodiments, a subject has or is at risk for developing cancer.

In some embodiments, the present disclosure provides methods of enhancing an immune response in a subject in need thereof, the method comprising administering to the subject a composition that comprises or delivers a T cell comprising a GPC3 CAR expressing IL-18. In some embodiments, a T cell comprising a GPC3 CAR expressing IL-18 is an autologous T cell. In some embodiments, the present disclosure provides methods of enhancing an immune response in a subject in need thereof, the method comprising administering to the subject a composition that comprises or delivers a T cell comprising a nucleic acid and/or vector encoding a GPC3 CAR expressing IL-18. In some embodiments a T cell comprising a nucleic acid and/or vector encoding a GPC3 CAR expressing IL-18 is an autologous T cell. In some embodiments, a subject has or is at risk for developing cancer.

In some embodiments, a disease suitable for treatment with compositions and methods of the present disclosure is selected from a proliferative disease such as a cancer or malignancy or a precancerous condition. In some embodiments, a disease is associated with expression of GPC3. In some embodiments, a disease suitable for treatment with compositions and methods of the present disclosure is a cancer. In some embodiments, a cancer expresses a GPC3 antigen. In some embodiments, a cancer cell has increased expression of GPC3 antigen relative to a non-cancer cell from a subject. In some embodiments, GPC3 expression levels can increase in a subject with cancer. In some embodiments, GPC3 expression levels can be undetectable in a healthy subject.

### Pharmaceutical Compositions

In some embodiments, the present disclosure provides pharmaceutical compositions that include a T cell comprising a GPC3 CAR expressing IL-18 and a pharmaceutically acceptable carrier. In some embodiments, a T cell comprising a GPC3 CAR expressing IL-18 is an autologous T cell. In some embodiments, the present disclosure provides pharmaceutical compositions that include a T cell comprising a nucleic acid and/or vector encoding a GPC3 CAR expressing IL-18 and a pharmaceutically acceptable carrier. In some embodiments a T cell comprising a nucleic acid and/or vector encoding a GPC3 CAR expressing IL-18 is an autologous T cell. Compositions of the present disclosure include pharmaceutical compositions that include a T cell comprising a GPC3 CAR expressing IL-18 and/or a nucleic acid encoding a GPC3 CAR expressing IL-18 obtained by a method disclosed herein. In some embodiments, a pharmaceutical composition can include a buffer, a diluent, solubilizer, emulsifier, preservative, adjuvant, an excipient, or any combination thereof. In some embodiments, a composition, if desired, can also contain one or more additional therapeutically active substances.

In some embodiments, T cells of the present disclosure are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

In some embodiments, compositions are formulated for parenteral administration. For example, a pharmaceutical composition provided herein may be provided in a sterile injectable form (e.g., a form that is suitable for subcutaneous injection, hepatic artery infusion, or intravenous infusion). For example, in some embodiments, a pharmaceutical composition is provided in a liquid dosage form that is suitable for injection. In some embodiments, a pharmaceutical composition is provided as powders (e.g., lyophilized and/or sterilized), optionally under vacuum, which can be reconstituted with an aqueous diluent (e.g., water, buffer, salt solution, etc.) prior to injection. In some embodiments, a pharmaceutical composition is diluted and/or reconstituted in water, sodium chloride solution, sodium acetate solution, benzyl alcohol solution, phosphate buffered saline, etc. In some embodiments, a powder should be mixed gently with the aqueous diluent (e.g., not shaken).

In some embodiments, a T cell comprising a GPC3 CAR expressing IL-18 and/or a nucleic acid encoding a GPC3 CAR expressing IL-18 of the present disclosure is formulated with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. A vehicle or lyophilized powder can contain additives that maintain isotonicity (e.g., sodium chloride, mannitol) and chemical stability (e.g., buffers and preservatives). In some embodiments, a formulation is sterilized by known or suitable techniques. A pharmaceutical composition may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a composition including a population of T cells comprising a GPC3 CAR expressing IL-18 and/or a nucleic acid encoding a GPC3 CAR expressing IL-18 of the present disclosure is stably formulated. In some embodiments, a stable formulation of a population of T cells comprising a GPC3 CAR expressing IL-18 and/or a nucleic acid encoding a GPC3 CAR expressing IL-18 of the present disclosure may comprise a phosphate buffer with saline or a chosen salt, as well as preserved solutions and formulations containing a preservative as well as multi-use preserved formulations suitable for pharmaceutical or veterinary use. Preserved formulations contain at least one known preservative or optionally selected from the group consisting of at least one phenol, m-cresol, pcresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

In some embodiments, a pharmaceutical composition is provided in a form that can be refrigerated and/or frozen. In some embodiments, a pharmaceutical composition is provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of compositions including an antibody agent for longer than the specified time results in degradation of the antibody agent. Liquid dosage forms and/or reconstituted solutions may comprise particulate matter and/or discoloration prior to administration. In some embodiments, a solution should not be used if discolored or cloudy and/or if particulate matter remains after filtration. General considerations in the formulation and/or manufacture of pharmaceutical agents may be found, for example, in Remington: The Science and Practice of Pharmacy 21st ed., Lippincott Williams & Wilkins, 2005.

In some embodiments, a pharmaceutical composition including a T cell comprising a GPC3 CAR expressing IL-18 and/or a nucleic acid encoding a GPC3 CAR expressing IL-18 of the present disclosure can be included in a container for storage or administration, for example, a vial, a syringe (e.g., an IV syringe), or a bag (e.g., an IV bag). A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

### Kits

The present disclosure further provides a kit comprising one or more containers filled with at least one GPC3 CAR expressing IL-18 and/or a nucleic acid encoding a GPC3 CAR expressing IL-18 as described herein. Kits may be used in any applicable method, including, for example, therapeutic methods, diagnostic methods, cell proliferation and/or isolation methods, etc. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In some embodiments, a kit may include one or more reagents for detection (e.g., detection of a GPC3 CAR expressing IL-18 and/or a nucleic acid encoding a GPC3 CAR expressing IL-18. In some embodiments, a kit may include a GPC3 CAR expressing IL-18 and/or a nucleic acid encoding a GPC3 CAR expressing IL-18 in a detectable form (e.g., covalently associated with detectable moiety or entity). In some embodiments, one or more GPC3 CARs expressing IL-18 and/or a nucleic acid encoding a GPC3 CAR expressing IL-18 as provided herein may be included in a kit used for treatment of subjects. In some embodiments, a GPC3 CAR expressing IL-18 and/or a nucleic acid encoding a GPC3 CAR expressing IL-18 as provided herein may be included in a kit used for preparing an autologous T cell expressing the GPC3 CAR expressing IL-18.

In some embodiments, a kit may provide one, two, three, four or more GPC3 antibody agents, where each is suitable for cloning into a CAR construct. In some embodiments, a kit may provide other reagents for assaying binding affinity of a GPC3 antibody agent and/or GPC3 CAR expressing IL-18 and/or a GPC3 CAR T cell expressing IL-18 for a T cell or GPC3 identified or isolated from a subject. In some embodiments, a kit may provide other reagents for assaying functional avidity of an antibody agent and/or GPC3 CAR expressing IL-18 and/or a GPC3 CAR T cell expressing IL-18 for a T cell of a subject.

A number of embodiments have been described. Nevertheless, it will be understood that various modifications may be made without departing form the spirit and scope of the invention.

### EXEMPLARY EMBODIMENTS

Embodiment 1. An immune cell comprising:
a chimeric antigen receptor (CAR), wherein the CAR comprises an extracellular antigen-binding domain that binds specifically to glypican-3 (GPC3), a transmembrane domain, and an intracellular signaling domain; and an exogenous nucleic acid comprising a sequence encoding interleukin-18.
Embodiment 2. The immune cell of embodiment 1, wherein the interleukin-18 is a human interleukin-18.
Embodiment 3. The immune cell of embodiment 2, wherein the human interleukin-18 comprises a sequence that is at least 80% identical to SEQ ID NO: 11 or 12.
Embodiment 4. The immune cell of embodiment 3, wherein the human interleukin-18 comprises a sequence that is at least 90% identical to SEQ ID NO: 11 or 12.
Embodiment 5. The immune cell of embodiment 4, wherein the human interleukin-18 comprises a sequence that is at least 96% identical to SEQ ID NO: 11 or 12.
Embodiment 6. The immune cell of any one of embodiments 1-5, wherein the sequence encoding interleukin-18 further includes a sequence encoding a secretion signal sequence.
Embodiment 7. The immune cell of embodiment 6, wherein the secretion signal sequence is an interleukin-2 secretion signal sequence.
Embodiment 8. The immune cell of embodiment 7, wherein the interleukin-2 secretion signal sequence comprises a sequence of SEQ ID NO: 13 or 14.
Embodiment 9. The immune cell of any one of embodiments 1-8, wherein the exogenous nucleic acid further comprises a promoter operably linked to the sequence encoding interleukin-18.
Embodiment 10. The immune cell of embodiment 9, wherein the promoter is a constitutive promoter.
Embodiment 11. The immune cell of embodiment 9, wherein the promoter is an inducible promoter.
Embodiment 12. The immune cell of embodiment 9, wherein the promoter is an NFAT promoter.
Embodiment 13. The immune cell of any one of embodiments 1-12, wherein the exogenous nucleic acid further comprises a sequence encoding the CAR.
Embodiment 14. The immune cell of embodiment 13, wherein the exogenous nucleic acid further comprises a promoter operably linked to the sequence encoding the CAR.
Embodiment 15. The immune cell of embodiment 14, wherein the promoter is a constitutive promoter.
Embodiment 16. The immune cell of embodiment 14, wherein the promoter is an inducible promoter.
Embodiment 17. The immune cell of embodiment 14, wherein the promoter is an NFAT promoter.
Embodiment 18. The immune cell of any one of embodiments 1-17, wherein the CAR is a single polypeptide.
Embodiment 19. The immune cell of any one of embodiments 1-17, wherein the CAR is comprised of two polypeptides.
Embodiment 20. The immune cell of any one of embodiments 1-19, wherein the extracellular antigen-binding domain comprises: a light chain variable domain comprising a CDR1 comprising SEQ ID NO: 1, a CDR2 comprising SEQ ID NO: 2, and a CDR3 comprising SEQ ID NO: 3; and a heavy chain variable domain comprising a CDR1 comprising SEQ ID NO: 4, a CDR2 comprising SEQ ID NO: 5, and a CDR3 comprising SEQ ID NO: 6.
Embodiment 21. The immune cell of embodiment 20, wherein the light chain variable domain comprises a sequence that is at least 80% identical to SEQ ID NO: 10.
Embodiment 22. The immune cell of embodiment 21, wherein the light chain variable domain comprises a sequence that is at least 90% identical to SEQ ID NO: 10.
Embodiment 23. The immune cell of embodiment 22, wherein the light chain variable domain comprises a sequence that is at least 96% identical to SEQ ID NO: 10.
Embodiment 24. The immune cell of any one of embodiments 20-23, wherein the heavy chain variable domain comprises a sequence that is at least 80% identical to SEQ ID NO: 8.
Embodiment 25. The immune cell of embodiment 24, wherein the heavy chain variable domain comprises a sequence that is at least 90% identical to SEQ ID NO: 8.
Embodiment 26. The immune cell of embodiment 25, wherein the heavy chain variable domain comprises a sequence that is at least 96% identical to SEQ ID NO: 8.
Embodiment 27. The immune cell of any one of embodiments 1-26, wherein the antigen-binding domain is humanized.
Embodiment 28. The immune cell of any one of embodiments 1-26, wherein the antigen-binding domain is human.
Embodiment 29. The immune cell of any one of embodiments 1-28, wherein the antigen-binding domain is an scFv.
Embodiment 30. The immune cell of any one of embodiments 1-29, wherein the transmembrane domain is a transmembrane domain selected from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6.
Embodiment 31. The immune cell of embodiment 30, wherein the transmembrane domain is a transmembrane domain from CD8alpha.
Embodiment 32. The immune cell of any one of embodiments 1-31, wherein the intracellular signaling domain comprises an intracellular signaling domain from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3epsilon, CD3gamma, CD3zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRTAM, a cytokine receptor, DAP-10, DNAM1 (CD226), Fc gamma receptor, GADS, GITR, HVEM (LIGHTR), IA4, ICAM-1, Ig alpha (CD79a), IL-2Rbeta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08, lymphocyte function-associated antigen-1 (LFA-1), a MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecules (SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6, or any combination thereof.
Embodiment 33. The immune cell of embodiment 32, wherein the intracellular signaling domain is from 4-1BB and CD3zeta.
Embodiment 34. The immune cell of any one of embodiments 1-33, wherein the chimeric antigen receptor further comprises an additional antigen-binding domain.
Embodiment 35. The immune cell of embodiment 34, wherein the additional antigen-binding domain is an scFv.
Embodiment 36. The immune cell of any one of embodiments 1-35, wherein the immune cell is a human immune cell.
Embodiment 37. The immune cell of embodiment 36, wherein the human immune cell is an autologous human immune cell.
Embodiment 38. The immune cell of embodiment 36, wherein the human immune cell is an allogeneic human immune cell.
Embodiment 39. The immune cell of any one of embodiments 1-38, wherein the immune cell is a T cell.
Embodiment 40. The immune cell of any one of embodiments 1-38, wherein the immune cell is an NK cell.
Embodiment 41. The immune cell of any one of embodiments 1-40, wherein the immune cell secretes the IL-18 encoded by the exogenous nucleic acid.
Embodiment 42. A pharmaceutical composition comprising an immune cell of any one of embodiments 1-41 and a pharmaceutically acceptable carrier.
Embodiment 43. A kit comprising a pharmaceutical composition of embodiment 42.
Embodiment 44. A method of treating a subject having a glypican-3-associated cancer, the method comprising administering to the subject an immune cell of any one of embodiments 1-41 or a pharmaceutical composition of embodiment 42.
Embodiment 45. A nucleic acid comprising: a sequence encoding a chimeric antigen receptor (CAR), wherein the CAR comprises: an extracellular antigen-binding domain that binds specifically to glypican-3 (GPC3), a transmembrane domain, and an intracellular signaling domain; and a sequence encoding an interleukin-18.
Embodiment 46. The nucleic acid of embodiment 45, wherein the interleukin-18 is a human interleukin-18.
Embodiment 47. The nucleic acid of embodiment 46, wherein the human interleukin-18 comprises a sequence that is at least 80% identical to SEQ ID NO: 11 or 12.
Embodiment 48. The nucleic acid of embodiment 47, wherein the human interleukin-18 comprises a sequence that is at least 90% identical to SEQ ID NO: 11 or 12.
Embodiment 49. The nucleic acid of embodiment 48, wherein the human interleukin-18 comprises a sequence that is at least 96% identical to SEQ ID NO: 11 or 12.
Embodiment 50. The nucleic acid of any one of embodiments 45-49, wherein the sequence encoding interleukin-18 further comprises a sequence encoding a secretion signal sequence.
Embodiment 51. The nucleic acid of embodiment 50, wherein the secretion signal sequence is an interleukin-2 secretion signal sequence.
Embodiment 52. The nucleic acid of embodiment 51, wherein the interleukin-2 secretion signal sequence comprises a sequence of SEQ ID NO: 13 or 14.
Embodiment 53. The nucleic acid of any one of embodiments 45-52, wherein the nucleic acid further comprises a promoter operably linked to the sequence encoding interleukin-18, wherein the promoter is disposed between the sequence encoding the CAR and the sequence encoding interleukin-18.
Embodiment 54. The nucleic acid of embodiment 53, wherein the promoter is a constitutive promoter.
Embodiment 55. The nucleic acid of embodiment 53, wherein the promoter is an inducible promoter.
Embodiment 56. The nucleic acid of embodiment 53, wherein the promoter is an NFAT promoter.
Embodiment 57. The nucleic acid of any one of embodiments 45-52, wherein the nucleic acid further includes a sequence encoding a self-cleaving protein sequence disposed between the sequence encoding the CAR and the sequence encoding interleukin-18.
Embodiment 58. The nucleic acid of embodiment 57, wherein the self-cleaving protein sequence is a P2 self-cleaving protein sequence.
Embodiment 59. The nucleic acid of any one of embodiments 45-58, wherein the nucleic acid further comprises a poly(A) sequence disposed between the sequence encoding the CAR and the sequence encoding interleukin-18.
Embodiment 60. The nucleic acid of any one of embodiments 45-59, wherein the nucleic acid further comprises a promoter operably linked to the sequence encoding the CAR.
Embodiment 61. The nucleic acid of embodiment 60, wherein the promoter is a constitutive promoter.
Embodiment 62. The nucleic acid of embodiment 60, wherein the promoter is an inducible promoter.
Embodiment 63. The nucleic acid of embodiment 60, wherein the promoter is an NFAT promoter.
Embodiment 64. The nucleic acid of any one of embodiments 45-63, wherein the CAR is a single polypeptide.
Embodiment 65. The nucleic acid of any one of embodiments 45-63, wherein the CAR is comprised of two polypeptides.
Embodiment 66. The nucleic acid of any one of embodiments 45-65, wherein the extracellular antigen-binding domain comprises: a light chain variable domain comprising a CDR1 comprising SEQ ID NO: 1, a CDR2 comprising SEQ ID NO: 2, and a CDR3 comprising SEQ ID NO: 3; and a heavy chain variable domain comprising a CDR1 comprising SEQ ID NO: 4, a CDR2 comprising SEQ ID NO: 5, and a CDR3 comprising SEQ ID NO: 6.
Embodiment 67. The nucleic acid of embodiment 66, wherein the light chain variable domain comprises a sequence that is at least 80% identical to SEQ ID NO: 10.
Embodiment 68. The nucleic acid of embodiment 67, wherein the light chain variable domain comprises a sequence that is at least 90% identical to SEQ ID NO: 10.
Embodiment 69. The nucleic acid of embodiment 68, wherein the light chain variable domain comprises a sequence that is at least 96% identical to SEQ ID NO: 10.
Embodiment 70. The nucleic acid of any one of embodiments 66-69, wherein the heavy chain variable domain comprises a sequence that is at least 80% identical to SEQ ID NO: 8.
Embodiment 71. The nucleic acid of embodiment 70, wherein the heavy chain variable domain comprises a sequence that is at least 90% identical to SEQ ID NO: 8.
Embodiment 72. The nucleic acid of embodiment 71, wherein the heavy chain variable domain comprises a sequence that is at least 96% identical to SEQ ID NO: 8.
Embodiment 73. The nucleic acid of any one of embodiments 45-72, wherein the antigen-binding domain is humanized.
Embodiment 74. The nucleic acid of any one of embodiments 45-72, wherein the antigen-binding domain is human.
Embodiment 75. The nucleic acid of any one of embodiments 45-74, wherein the antigen-binding domain is an scFv.
Embodiment 76. The nucleic acid of any one of embodiments 45-75, wherein the transmembrane domain is a transmembrane domain selected from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6.
Embodiment 77. The nucleic acid of embodiment 76, wherein the transmembrane domain is a transmembrane domain from CD8alpha.
Embodiment 78. The nucleic acid of any one of embodiments 45-77, wherein the intracellular signaling domain comprises an intracellular signaling domain from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3epsilon, CD3gamma, CD3zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRTAM, a cytokine receptor, DAP-10, DNAM1 (CD226), Fc gamma receptor, GADS, GITR, HVEM (LIGHTR), IA4, ICAM-1, Ig alpha (CD79a), IL-2Rbeta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08, lymphocyte function-associated antigen-1 (LFA-1), a MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecules (SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6, or any combination thereof.
Embodiment 79. The nucleic acid of embodiment 78, wherein the intracellular signaling domain is from 4-1BB and CD3zeta.
Embodiment 80. The nucleic acid of any one of embodiments 45-79, wherein the chimeric antigen receptor further comprises an additional antigen-binding domain.
Embodiment 81. The nucleic acid of embodiment 80, wherein the additional antigen-binding domain is an scFv.
Embodiment 82. A vector comprising the nucleic acid of any one of embodiments 45-81.
Embodiment 83. The vector of embodiment 82, wherein the vector is a viral vector.
Embodiment 84. The vector of embodiment 83, wherein the viral vector is a lentiviral vector.
Embodiment 85. A method of producing an engineered immune cell, the method comprising: introducing into an immune cell a nucleic acid of any one of embodiments 45-81 or a vector of any one of embodiments 82-84, thereby producing the engineered immune cell.
Embodiment 86. The method of embodiment 85, further comprising, after the introducing step, culturing the engineered immune cell.
Embodiment 87. The method of embodiment 85 or 86, wherein the immune cell is a T cell.
Embodiment 88. The method of embodiment 85 or 86, wherein the immune cell is a NK cell.
Embodiment 89. The method of any one of embodiments 85-88, further comprising, before the introducing step, obtaining the immune cell from a subject.
Embodiment 90. The method of embodiment 89, wherein the method further comprises administering the engineered immune cell to the subject.
Embodiment 91. The method of embodiment 89 or 90, wherein the subject has been diagnosed or identified as having a glypican-3-associated cancer.
Embodiment 92. An engineered immune cell produced by the method of any one of embodiments 85-89.
Embodiment 93. A pharmaceutical composition comprising the engineered immune cell of embodiment 92 and a pharmaceutically acceptable carrier.
Embodiment 94. A method of treating an anti-glypican-3-associated cancer in a subject, the method comprising administering to the subject an engineered immune cell of embodiment 92 or a pharmaceutical composition of embodiment 93.
Embodiment 95. A pair of nucleic acids comprising: a first nucleic acid comprising a sequence encoding a chimeric antigen receptor (CAR), wherein the CAR comprises: an extracellular antigen-binding domain that binds specifically to glypican-3 (GPC3), a transmembrane domain, and an intracellular signaling domain; and a second nucleic acid comprising a sequence encoding an interleukin-18.
Embodiment 96. The pair of nucleic acids of embodiment 95, wherein the interleukin-18 is a human interleukin-18.
Embodiment 97. The pair of nucleic acids of embodiment 96, wherein the human interleukin-18 comprises a sequence that is at least 80% identical to SEQ ID NO: 11 or 12.
Embodiment 98. The pair of nucleic acids of embodiment 97, wherein the human interleukin-18 comprises a sequence that is at least 90% identical to SEQ ID NO: 11 or 12.
Embodiment 99. The pair of nucleic acids of embodiment 98, wherein the human interleukin-18 comprises a sequence that is at least 96% identical to SEQ ID NO: 11 or 12.
Embodiment 100. The pair of nucleic acids of any one of embodiments 95-99, wherein the sequence encoding interleukin-18 further comprises a sequence encoding a secretion signal sequence.
Embodiment 101. The pair of nucleic acids of embodiment 100, wherein the secretion signal sequence is an interleukin-2 secretion signal sequence.
Embodiment 102. The pair of nucleic acids of embodiment 101, wherein the interleukin-2 secretion signal sequence comprises a sequence of SEQ ID NO: 13 or 14.
Embodiment 103. The pair of nucleic acids of any one of embodiments 95-102, wherein the second nucleic acid further comprises a promoter operably linked to the sequence encoding interleukin-18.
Embodiment 104. The pair of nucleic acids of embodiment 103, wherein the promoter is a constitutive promoter.
Embodiment 105. The pair of nucleic acids of embodiment 103, wherein the promoter is an inducible promoter.
Embodiment 106. The pair of nucleic acids of embodiment 103, wherein the promoter is an NFAT promoter.
Embodiment 107. The pair of nucleic acids of any one of embodiments 95-106, wherein the first and/or second nucleic acid further comprises a poly(A) sequence.
Embodiment 108. The pair of nucleic acids of any one of embodiments 95-107, wherein the first nucleic acid further comprises a promoter operably linked to the sequence encoding the CAR.
Embodiment 109. The pair of nucleic acids of embodiment 108, wherein the promoter is a constitutive promoter.
Embodiment 110. The pair of nucleic acids of embodiment 108, wherein the promoter is an inducible promoter.
Embodiment 111. The pair of nucleic acids of embodiment 108, wherein the promoter is an NFAT promoter.
Embodiment 112. The pair of nucleic acids of any one of embodiments 95-111, wherein the CAR is a single polypeptide.
Embodiment 113. The pair of nucleic acids of any one of embodiments 95-111, wherein the CAR is comprised of two polypeptides.
Embodiment 114. The pair of nucleic acids of any one of embodiments 95-113, wherein the extracellular antigen-binding domain comprises: a light chain variable domain comprising a CDR1 comprising SEQ ID NO: 1, a CDR2 comprising SEQ ID NO: 2, and a CDR3 comprising SEQ ID NO: 3; and a heavy chain variable domain comprising a CDR1 comprising SEQ ID NO: 4, a CDR2 comprising SEQ ID NO: 5, and a CDR3 comprising SEQ ID NO: 6.
Embodiment 115. The pair of nucleic acids of embodiment 114, wherein the light chain variable domain comprises a sequence that is at least 80% identical to SEQ ID NO: 10.
Embodiment 116. The pair of nucleic acids of embodiment 115, wherein the light chain variable domain comprises a sequence that is at least 90% identical to SEQ ID NO: 10.
Embodiment 117. The pair of nucleic acids of embodiment 116, wherein the light chain variable domain comprises a sequence that is at least 96% identical to SEQ ID NO: 10.
Embodiment 118. The pair of nucleic acids of any one of embodiments 114-117, wherein the heavy chain variable domain comprises a sequence that is at least 80% identical to SEQ ID NO: 8.
Embodiment 119. The pair of nucleic acids of embodiment 118, wherein the heavy chain variable domain comprises a sequence that is at least 90% identical to SEQ ID NO: 8.
Embodiment 120. The pair of nucleic acids of embodiment 119, wherein the heavy chain variable domain comprises a sequence that is at least 96% identical to SEQ ID NO: 8.
Embodiment 121. The pair of nucleic acids of any one of embodiments 95-120, wherein the antigen-binding domain is humanized.
Embodiment 122. The pair of nucleic acids of any one of embodiments 95-120, wherein the antigen-binding domain is human.
Embodiment 123. The pair of nucleic acids of any one of embodiments 95-122, wherein the antigen-binding domain is an scFv.
Embodiment 124. The pair of nucleic acids of any one of embodiments 95-123, wherein the transmembrane domain is a transmembrane domain selected from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM11, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6.
Embodiment 125. The pair of nucleic acids of embodiment 124, wherein the transmembrane domain is a transmembrane domain from CD8alpha.
Embodiment 126. The pair of nucleic acids of any one of embodiments 95-125, wherein the intracellular signaling domain comprises an intracellular signaling domain from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3epsilon, CD3gamma, CD3zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRTAM, a cytokine receptor, DAP-10, DNAM1 (CD226), Fc gamma receptor, GADS, GITR, HVEM (LIGHTR), IA4, ICAM-1, Ig alpha (CD79a), IL-2Rbeta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08, lymphocyte function-associated antigen-1 (LFA-1), a MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecules (SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6, or any combination thereof.
Embodiment 127. The pair of nucleic acids of embodiment 126, wherein the intracellular signaling domain is from 4-1BB and CD3zeta.
Embodiment 128. The pair of nucleic acids of any one of embodiments 95-127, wherein the chimeric antigen receptor further comprises an additional antigen-binding domain.
Embodiment 129. The pair of nucleic acids of embodiment 128, wherein the additional antigen-binding domain is an scFv.
Embodiment 130. A pair of vectors that together comprise the pair of nucleic acids of any one of embodiments 95-129.
Embodiment 131. The pair of vectors of embodiment 130, wherein the pair of vectors is a pair of viral vectors.
Embodiment 132. The pair of vectors of embodiment 131, wherein the pair of viral vectors is a pair of lentiviral vectors.
Embodiment 133. A method of producing an engineered immune cell, the method comprising: introducing into an immune cell a pair of nucleic acids of any one of embodiments 95-129 or a pair of vectors of any one of embodiments 130-132, thereby producing the engineered immune cell.
Embodiment 134. The method of embodiment 133, further comprising, after the introducing step, culturing the engineered immune cell.
Embodiment 135. The method of embodiment 133 or 134, wherein the immune cell is a T cell.
Embodiment 136. The method of embodiment 133 or 134, wherein the immune cell is a NK cell.
Embodiment 137. The method of any one of embodiments 133-136, further comprising, before the introducing step, obtaining the immune cell from a subject.
Embodiment 138. The method of embodiment 137, wherein the method further comprises administering the engineered immune cell to the subject.
Embodiment 139. The method of embodiment 137 or 138, wherein the subject has been diagnosed or identified as having a glypican-3-associated cancer.
Embodiment 140. An engineered immune cell produced by the method of any one of embodiments 133-137.
Embodiment 141. A pharmaceutical composition comprising the engineered immune cell of embodiment 140 and a pharmaceutically acceptable carrier.
Embodiment 142. A method of treating an anti-glypican-3-associated cancer in a subject, the method comprising administering to the subject an engineered immune cell of embodiment 140 or a pharmaceutical composition of embodiment 141.
Embodiment 143. The method of any one of embodiments 44, 94, and 142, wherein the glypican-3-associated cancer is liver cancer.
Embodiment 144. The method of any one of embodiments 44, 94, 142, and 143, wherein the subject has previously been administered one or more additional anticancer therapies selected from the group consisting of: ionizing radiation, a chemotherapeutic agent, a therapeutic antibody, and a checkpoint inhibitor.
Embodiment 145. The method of any one of embodiments 44, 94, 142, and 143, wherein the subject is further administered one or more additional anticancer therapies selected from the group consisting of: ionizing radiation, a chemotherapeutic agent, a therapeutic antibody, and a checkpoint inhibitor.
Embodiment 146. The method of any one of embodiments 44, 94, and 142-145, wherein the subject has been identified or diagnosed as having the glypican-3-associated cancer.

### Examples

The disclosure is further described in the following examples, which do not limit the scope of the disclosure described in the claims.

### Example 1—Lentiviral Transfer Plasmid

A DNA construct encoding a single-chain variable fragment (scFv) form of a humanized anti-GPC3 antibody (clone: GC33) agent was generated by using an IDT vector and connecting the VL and VH regions, wherein the sequence is designed to comprise VH-VL orientation or VL-VH orientation, using standard DNA cloning techniques known to the art. The lentiviral transfer plasmids used herein are shown in Table 3 and nucleic acid sequence for huGC33 VH-VL is shown in Table 4.

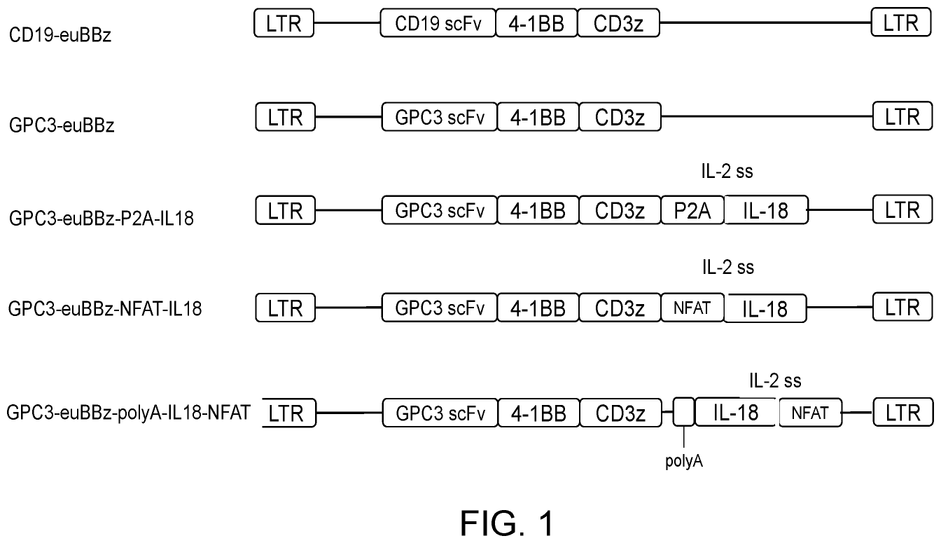

A GPC3 CAR expressing IL-18 was produced using a P2A self-cleaving peptide, or a nuclear factor of activated T cells (NFAT)-IL-2 minimal promoter was used to express IL-18 only in activated T cells (FIG. 1). The intracellular signaling domain of the GPC3 CARs expressing IL-18 includes a 4-1BB signaling domain followed by a five amino acid sequence. For the NFAT-IL-2 minimal promoter, the sequence from pGL3-NFAT luciferase was used, wherein the sequence information for NFAT-IL-2 minimal promoter-IL-18 and polyA-IL-18-NFAT-IL-2 minimal promoter is shown in Table 5.

TABLE 3
Lentiviral Transfer Vector

| | Intracellular | Selectable | Transfer |
|---|---|---|---|
| scFv | Domain | marker | vector |
| huGC33 VH-VL | euBBz | Flag | pELPS4 |
| huGC33 VH-VL | euBBz-P2A-IL18 | Flag | pELPS4 |
| huGC33 VH-VL | euBBz-NFAT-IL18 | Flag | pELPS4 |
| huGC33 VH-VL | euBBz-polyA-IL18-NFAT | Flag | pELPS4 |

TABLE 4

| | |
|---|---|
| huGC33 | CAAGTGCAACTCGTACAATCAGGTGCTGAAGTCAAAAAGCCG |
| VH-VL | GGAGCCTCTGTTAAAGTGTCCTGTAAAGCCAGCGGCTACACC |
| | TTTACCGATTATGAGATGCACTGGGTTCGGCAGGCTCCGGGC |
| | CAAGGTCTGGAGTGGATCGGGGCTCTTGACCCAAAGACGGGC |
| | GACACGGCTTATTCACAAAAATTCAAAGGTAGGGCTACTCTG |
| | ACTGCCGATAAGTCCACCAGCACCGCGTATATGGAGCTCTCT |
| | AGCTTGCGAAGCGAGGACACGGCGGTGTACTATTGCACACGC |
| | TTCTATAGTTACACATATTGGGGTCAAGGCACGCTTGTGACC |
| | GTGTCTAGCGGTGGCGGCGGAAGTGGTGGTGGTGGTTCTGGG |
| | GGCGGGGGTTCCGACGTCGTTATGACACAGAGTCCCCTCTCC |
| | TTGCCGGTGACCCTGGGTCAGCCTGCGTCCATCTCTTGCAGA |
| | TCCTCCCAGTCTCTGGTACACTCCAACGGCAACACATACTTG |
| | CACTGGTACCAACAAAGACCTGGTCAGTCACCGCGACTTCTC |
| | ATATATAAAGTTTCCAATAGGTTCAGTGGAGTGCCAGACAGG |
| | TTCAGTGGTTCAGGATCAGGCACTGATTTCACGCTTAAAATC |
| | AGTCGGGTTGAGGCGGAGGACGTAGGAGTTTACTATTGCAGC |
| | CAGAATACGCACGTGCCGCCTACTTTTGGCTCTGGAACCAAG |
| | TTGGAAATAAAG [SEQ ID NO: 15] |

TABLE 5

| | |
|---|---|
| euBBz-P2A-IL18 | [5A.A.+4-1BB] |
| | CGTTTCTCTGTTGTT (5 A.A) |
| | AAACGGGGCAGAAAGAAACTCCTGTATATATTCAAACAACCATTTA |
| | TGAGACCAGTACAAACTACTCAAGAGGAAGATGGCTGTAGCTGCCG |
| | ATTTCCAGAAGAAGAAGAAGGAGGATGTGAACTG (4-1BB) |
| | [CD3 Z] |
| | AGAGTGAAGTTCAGCAGGAGCGCAGACGCCCCCGCGTACAAGCAGG |
| | GCCAGAACCAGCTCTATAACGAGCTCAATCTAGGACGAAGAGAGGA |
| | GTACGATGTTTTGGACAAGAGACGTGGCCGGGACCCTGAGATGGGG |
| | GGAAAGCCGAGAAGGAAGAACCCTCAGGAAGGCCTGTACAATGAAC |
| | TGCAGAAAGATAAGATGGCGGAGGCCTACAGTGAGATTGGGATGAA |
| | AGGCGAGCGCCGGAGGGGCAAGGGGCACGATGGCCTTTACCAGGGT |
| | CTCAGTACAGCCACCAAGGACACCTACGACGCCCTTCACATGCAGG |
| | CCCTGCCCCCTCGC |
| | [P2A IL-18] |
| | GGC AGC GGC GCC ACA AAC TTC TCT CTG CTA AAG |
| | CAA GCA GGT GAT GTT GAA GAA AAC CCC GGG CCT |
| | (P2A) |
| | ATG TAC AGG ATG CAA CTC CTG TCT TGC ATT GCA |
| | CTA AGT CTT GCA CTT GTC ACA AAC AGT |
| | (IL-2 SS) |
| | TAC TTT GGC AAG CTT GAA TCT AAA TTA TCA GTC |
| | ATA AGA AAT TTG AAT GAC CAA GTT CTC TTC ATT |
| | GAC CAA GGA AAT CGG CCT CTA TTT GAA GAT ATG |
| | ACT GAT TCT GAC TGT AGA GAT AAT GCA CCC CGG |
| | ACC ATA TTT ATT ATA AGT ATG TAT AAA GAT AGC |
| | CAG CCT AGA GGT ATG GCT GTA ACT ATC TCT GTG |
| | AAG TGT GAG AAA ATT TCA ACT CTC TCC TGT GAG |
| | AAC AAA ATT ATT TCC TTT AAG GAA ATG AAT CCT |
| | CCT GAT AAC ATC AAG GAT ACA AAA AGT GAC ATC |
| | ATA TTC TTT CAG AGA AGT GTC CCA GGA CAT GAT |
| | AAT AAG ATG CAA TTT GAA TCT TCA TCA TAC GAA |
| | GGA TAC TTT CTA GCT TGT GAA AAA GAG AGA GAC |
| | CTT TTT AAA CTC ATT TTG AAA AAA GAG GAT GAA |
| | TTG GGG GAT AGA TCT ATA ATG TTC ACT GTT CAA |
| | AAC GAA GAC TAG (IL-18) |
| euBBz-NFAT-IL18 | [5A.A.+4-1BB] |
| | CGTTTCTCTGTTGTT (5A.A) |
| | AAACGGGGCAGAAAGAAACTCCTGTATATATTCAAACAACCATTTA |
| | TGAGACCAGTACAAACTACTCAAGAGGAAGATGGCTGTAGCTGCCG |
| | ATTTCCAGAAGAAGAAGAAGGAGGATGTGAACTG (4-1BB) |
| | [CD3 Z] |
| | AGAGTGAAGTTCAGCAGGAGCGCAGACGCCCCCGCGTACAAGCAGG |
| | GCCAGAACCAGCTCTATAACGAGCTCAATCTAGGACGAAGAGAGGA |
| | GTACGATGTTTTGGACAAGAGACGTGGCCGGGACCCTGAGATGGGG |
| | GGAAAGCCGAGAAGGAAGAACCCTCAGGAAGGCCTGTACAATGAAC |
| | TGCAGAAAGATAAGATGGCGGAGGCCTACAGTGAGATTGGGATGAA |
| | AGGCGAGCGCCGGAGGGGCAAGGGGCACGATGGCCTTTACCAGGGT |
| | CTCAGTACAGCCACCAAGGACACCTACGACGCCCTTCACATGCAGG |
| | CCCTGCCCCCTCGCTAA |
| | [NFAT IL-18]: |
| | ACGCCTTCTGTATGAAACAGTTTTTCCTCCACGCCTTCTGTATGAA |
| | ACAGTTTTTCCTCCACGCCTTCTGTATGAAACAGTTTTTCCTCCAC |
| | GCCTTCTGTATGAAACAGTTTTTCCTCCACGCCTTCTGTATGAAAC |
| | AGTTTTTCCTCCACGCCTTCTGTATGAAACAGTTTTTCCTCCTCGA |
| | GGACATTTTGACACCCCCATAATATTTTTCCAGAATTAACAGTATA |
| | AATTGCATCTCTTGTTCAAGAGTTCCCTATCACTCTCTTTAATCAC |
| | TACTCACAGTAACCTCAACTCCTGC |
| | (NFAT) |
| | ATGTACAGGATGCAACTCCTGTCTTGCATTGCACTAAGTCTTGCAC |
| | TTGTCACAAACAGT (IL-2 SS) |
| | TACTTTGGCAAGCTTGAATCTAAATTATCAGTCATAAGAAATTTGA |
| | ATGACCAAGTTCTCTTCATTGACCAAGGAAATCGGCCTCTATTTGA |
| | AGATATGACTGATTCTGACTGTAGAGATAATGCACCCCGGACCATA |
| | TTTATTATAAGTATGTATAAAGATAGCCAGCCTAGAGGTATGGCTG |
| | TAACTATCTCTGTGAAGTGTGAGAAAATTTCAACTCTCTCCTGTGA |
| | GAACAAAATTATTTCCTTTAAGGAAATGAATCCTCCTGATAACATC |
| | AAGGATACAAAAAGTGACATCATATTCTTTCAGAGAAGTGTCCCAG |
| | GACATGATAATAAGATGCAATTTGAATCTTCATCATACGAAGGATA |
| | CTTTCTAGCTTGTGAAAAAGAGAGAGACCTTTTTAAACTCATTTTG |
| | AAAAAAGAGGATGAATTGGGGGATAGATCTATAATGTTCACTGTTC |
| | AAAACGAAGACTAG (IL-18) |
| euBBz-polyA- | [5A.A.+4-1BB] |
| IL18-NFAT | CGTTTCTCTGTTGTT (5A.A) |
| | AAACGGGGCAGAAAGAAACTCCTGTATATATTCAAACAACCATTTA |
| | TGAGACCAGTACAAACTACTCAAGAGGAAGATGGCTGTAGCTGCCG |
| | ATTTCCAGAAGAAGAAGAAGGAGGATGTGAACTG (4-1BB) |
| | [CD3 Z] |
| | AGAGTGAAGTTCAGCAGGAGCGCAGACGCCCCCGCGTACAAGCAGG |
| | GCCAGAACCAGCTCTATAACGAGCTCAATCTAGGACGAAGAGAGGA |
| | GTACGATGTTTTGGACAAGAGACGTGGCCGGGACCCTGAGATGGGG |
| | GGAAAGCCGAGAAGGAAGAACCCTCAGGAAGGCCTGTACAATGAAC |
| | TGCAGAAAGATAAGATGGCGGAGGCCTACAGTGAGATTGGGATGAA |
| | AGGCGAGCGCCGGAGGGGCAAGGGGCACGATGGCCTTTACCAGGGT |
| | CTCAGTACAGCCACCAAGGACACCTACGACGCCCTTCACATGCAGG |
| | CCCTGCCCCCTCGCTAA |
| | [poly A IL-18 NFAT]: |
| | CAGACATGATAAGATACATTGATGAGTTTGGACAAACCACAACTAG |
| | AATGCAGTGAAAAAAATGCTTTATTTGTGAAATTTGTGATGCTATT |
| | GCTTTATTTGTAACCATTATAAGCTGCAATAAACAAGTTAACAACA |
| | ACAATTGCATTCATTTTATGTTTCAGGTTCAGGGGGAGGTGTGGGA |
| | GGTTTTTTAAAGCAAGTAAAACCTCTACAAATGTGGTATGGCTGAT |
| | TATGATC (Poly A) |
| | CTAGTCTTCGTTTTGAACAGTGAACATTATAGATCTATCCCCCAAT |
| | TCATCCTCTTTTTTCAAAATGAGTTTAAAAAGGTCTCTCTCTTTTT |
| | CACAAGCTAGAAAGTATCCTTCGTATGATGAAGATTCAAATTGCAT |
| | CTTATTATCATGTCCTGGGACACTTCTCTGAAAGAATATGATGTCA |
| | CTTTTTGTATCCTTGATGTTATCAGGAGGATTCATTTCCTTAAAGG |
| | AAATAATTTTGTTCTCACAGGAGAGAGTTGAAATTTTCTCACACTT |
| | CACAGAGATAGTTACAGCCATACCTCTAGGCTGGCTATCTTTATAC |
| | ATACTTATAATAAATATGGTCCGGGGTGCATTATCTCTACAGTCAG |
| | AATCAGTCATATCTTCAAATAGAGGCCGATTTCCTTGGTCAATGAA |
| | GAGAACTTGGTCATTCAAATTTCTTATGACTGATAATTTAGATTCA |
| | AGCTTGCCAAAGTA (IL-18) |
| | ACTGTTTGTGACAAGTGCAAGACTTAGTGCAATGCAAGACAGGAGT |
| | TGCATCCTGTACAT (IL-2 SS) |
| | GGAATTCAGGAGTTGAGGTTACTGTGAGTAGTGATTAAAGAGAGTG |
| | ATAGGGAACTCTTGAACAAGAGATGCAATTTATACTGTTAATTCTG |
| | GAAAAATATTATGGGGGTGTCAAAATGTCCCGGGGACGCCTTCTGT |
| | ATGAAACAGTTTTTCCTCCACGCCTTCTGTATGAAACAGTTTTTCC |
| | TCCACGCCTTCTGTATGAAACAGTTTTTCCTCCACGCCTTCTGTAT |
| | GAAACAGTTTTTCCTCCACGCCTTCTGTATGAAACAGTTTTTCCTC |
| | CACGCCTTCTGTATGAAACAGTTTTTCCTCC (NFAT) |

additional 5 amino acids
SEQ ID NO: 16
CGTTTCTCTGTTGTT
4-1BB intracellular domain
SEQ ID NO: 17
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG
4-1BB intracellular domain with
five additional amino acids (euBBz)
SEQ ID NO: 18
CGTTTCTCTGTTGTTAAACGGGGCAGAAAGAAACTCCTGTATATATTCAA
ACAACCATTTATGAGACCAGTACAAACTACTCAAGAGGAAGATGGCTGTA
GCTGCCGATTTCCAGAAGAAGAAGAAGGAGGATGTGAACTG
CD3-zeta
SEQ ID NO: 19
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA
P2A
SEQ ID NO: 20
GGC AGC GGC GCC ACA AAC TTC TCT CTG CTA AAG CAA
GCA GGT GAT GTT GAA GAA AAC CCC GGG CCT
NFAT
SEQ ID NO: 21
ACGCCTTCTGTATGAAACAGTTTTTCCTCCACGCCTTCTGTATGAAACAG
TTTTTCCTCCACGCCTTCTGTATGAAACAGTTTTTCCTCCACGCCTTCTG
TATGAAACAGTTTTTCCTCCACGCCTTCTGTATGAAACAGTTTTTCCTCC
ACGCCTTCTGTATGAAACAGTTTTTCCTCCTCGAGGACATTTTGACACCC
CCATAATATTTTTCCAGAATTAACAGTATAAATTGCATCTCTTGTTCAAG
AGTTCCCTATCACTCTCTTTAATCACTACTCACAGTAACCTCAACTCCTG
C
poly A
SEQ ID NO: 22
CAGACATGATAAGATACATTGATGAGTTTGGACAAACCACAACTAGAATG
CAGTGAAAAAAATGCTTTATTTGTGAAATTTGTGATGCTATTGCTTTATT
TGTAACCATTATAAGCTGCAATAAACAAGTTAACAACAACAATTGCATTC
ATTTTATGTTTCAGGTTCAGGGGGAGGTGTGGGAGGTTTTTTAAAGCAAG
TAAAACCTCTACAAATGTGGTATGGCTGATTATGATC

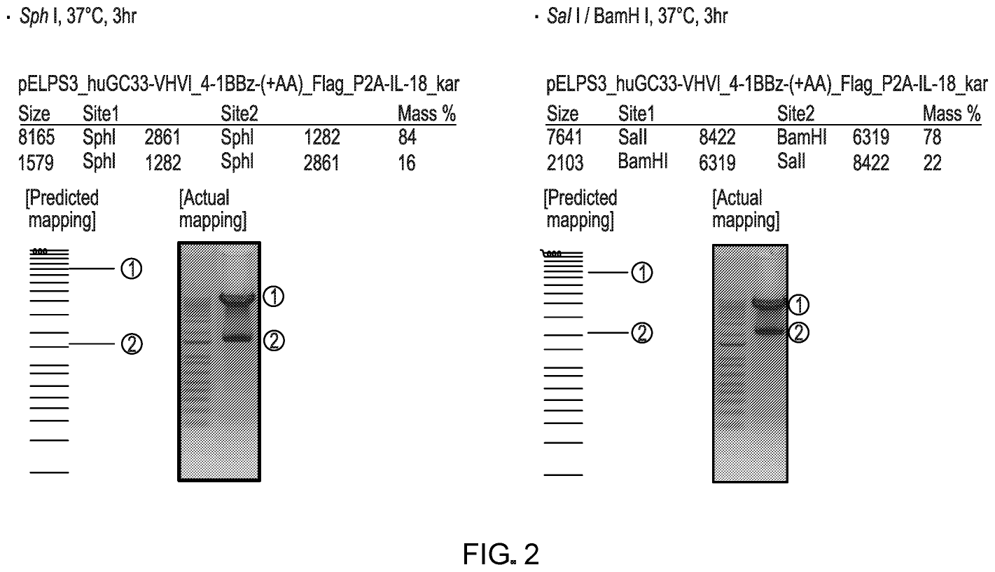

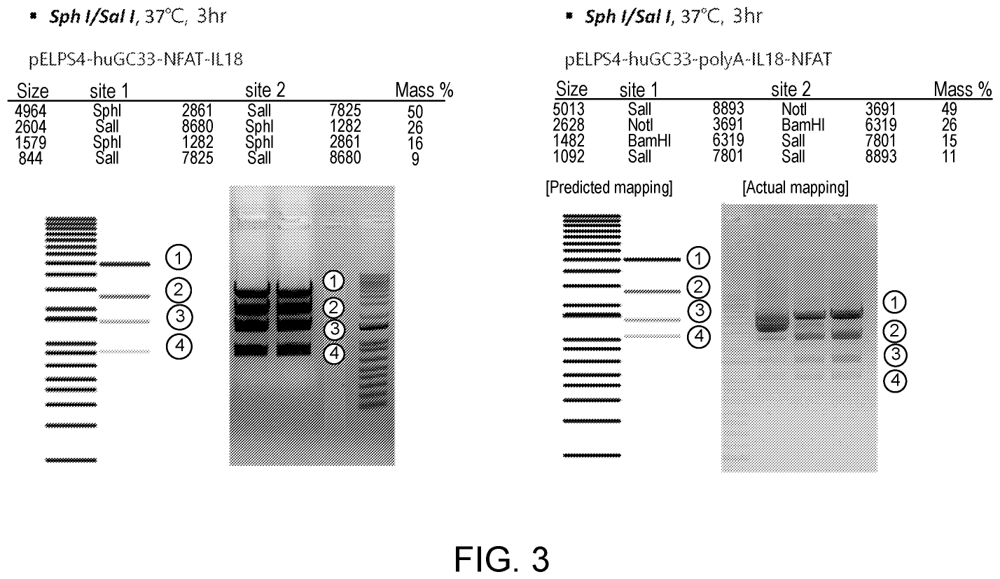

The lentivirus vector construct, pELPS4-huGC33-euBBz was digested with EcoRV and SalI, and pELPS4 huGC33 HL-P2A-IL18 was inserted into the vector construct. Furthermore, infusion cloning was performed for lentivirus vector constructs pELPS4-huGC33-euBBz-NFAT-IL18 and pELPS4-huGC33-euBBz-polyA-IL18-NFAT after the constructs were linearized with the SalI of the pELPS4-huGC33-euBBz, wherein DNA fragment purification results are shown in FIGS. 2 and 3. Transduction units (TU/mL) were measured for the lentiviruses and shown in Table. 6.

TABLE 6

| | | | | Transduction Units | |
|---|---|---|---|---|---|
| | | Select- | | (TU/mL) | |
| | Intracellular | able | Transfer | Production | Measure- |
| scFv | Domain | marker | vector | Date | ments |
| huGC33 | euBBz | Flag | pELPS4 | 2019 Sep. 24 | 3.58 × 10⁸ |
| VH-VL | | | | 2019 Oct. 21 | 5.22 × 10⁸ |
| huGC33 | euBBz-P2A- | Flag | pELPS4 | 2019 Nov. 25 | 8.51 × 10⁷ |
| VH-VL | IL18 | | | | |
| huGC33 | euBBz-NFAT- | Flag | pELPS4 | 2019 Nov. 5 | 1.64 × 10⁹ |
| VH-VL | IL18 | | | | |
| huGC33 | euBBz-polyA- | Flag | pELPS4 | 2020 Feb. 4 | 5.17 × 10⁸ |
| VH-VL | IL18-NFAT | | | | |
| CD19 | euBBz | Flag | pELPS4 | 2020 Feb. 11 | 2.37 × 10⁹ |

### Example 2—huGC33-VHVL-P2A-IL18 and huGC33-VHVL-NFAT-IL18 In Vitro

Peripheral blood mononuclear cells (PBMCs) were cultured in a cell culture medium including 1 L OpTmizer™ T-Cell Expansion Basal Medium, 25 mL OpTmizer™ T-Cell Expansion Supplement, 50 mL CTS™ Immune Cell SR, 10 mL Pen-Strep (10,000 U/mL), and 10 mL CTS™ GlutaMAX™-I Supplement, wherein cell density was adjusted to 1×10⁶ cells/mL, and IL-2 (400 IU/mL) was added during culture in an incubator at CO₂ 5% and 37° C.

The PBMCs were then transduced with the lentiviral vector and cell growth and expansion was measured from day 7 to day 14 of cell culture. The cells were then harvested on day 14 and used for further analysis.

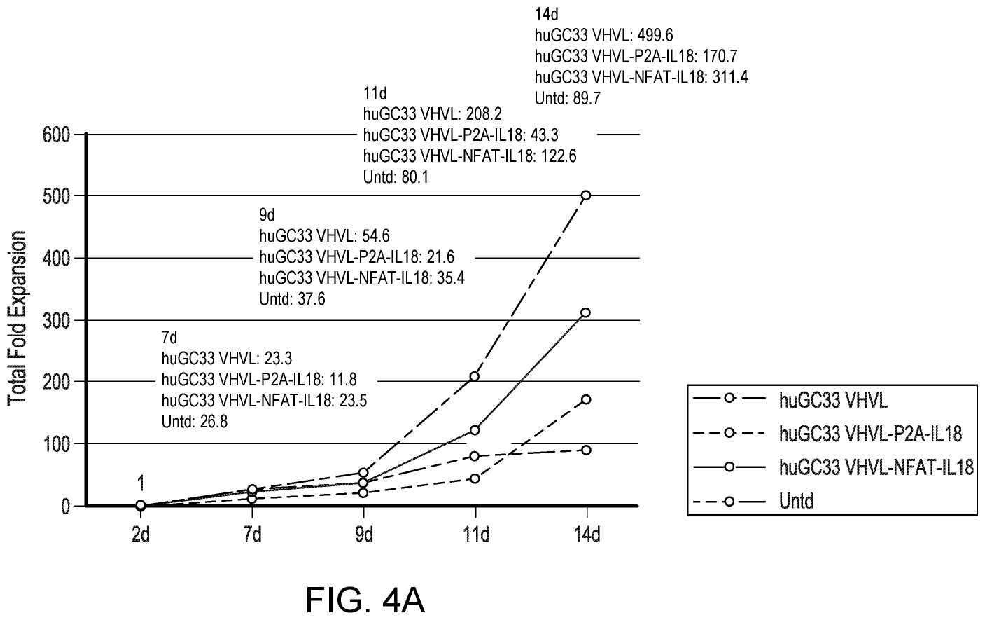

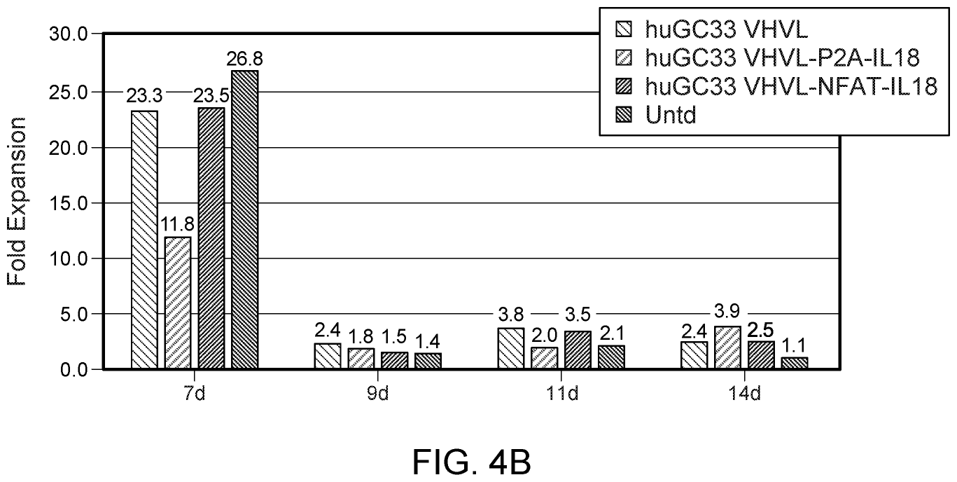

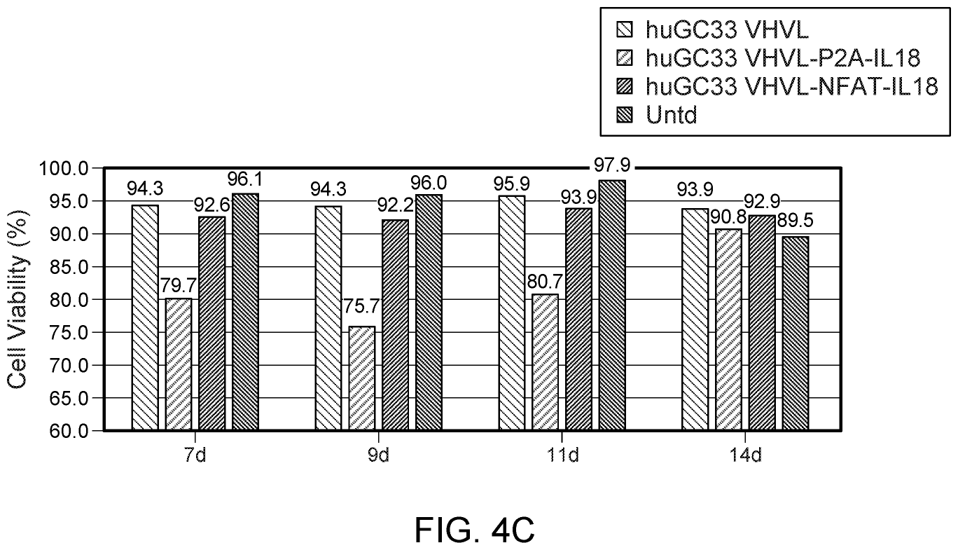

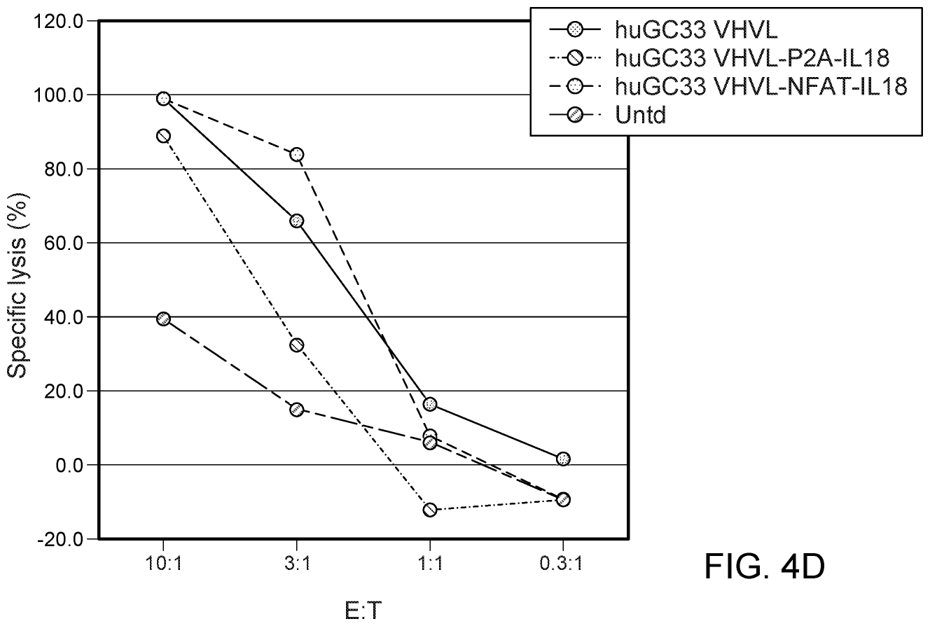

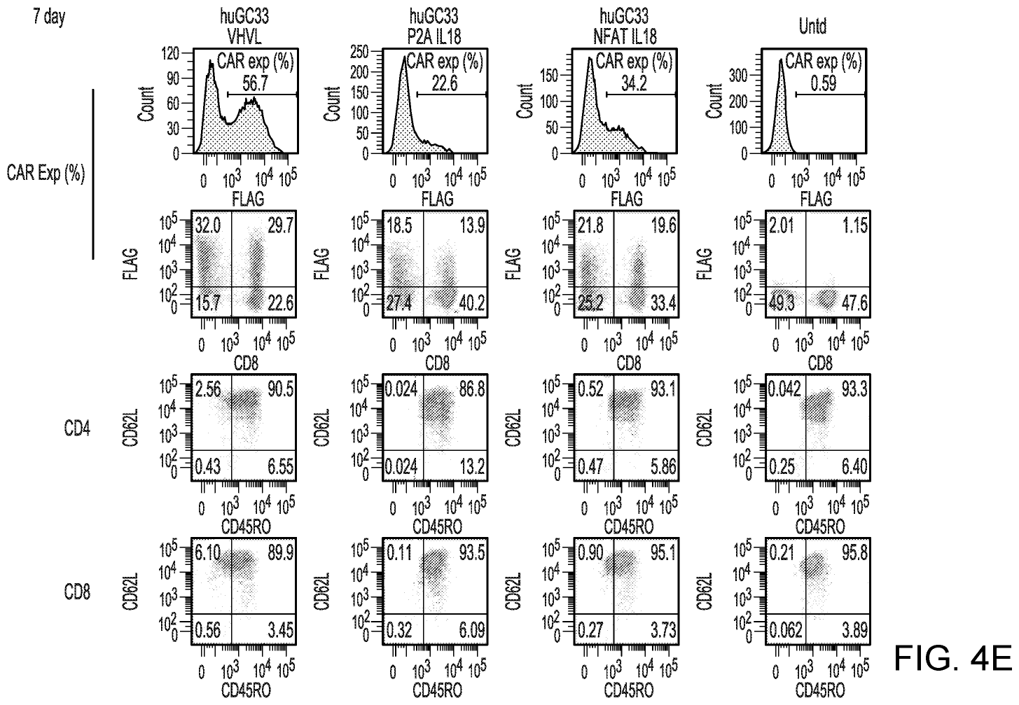

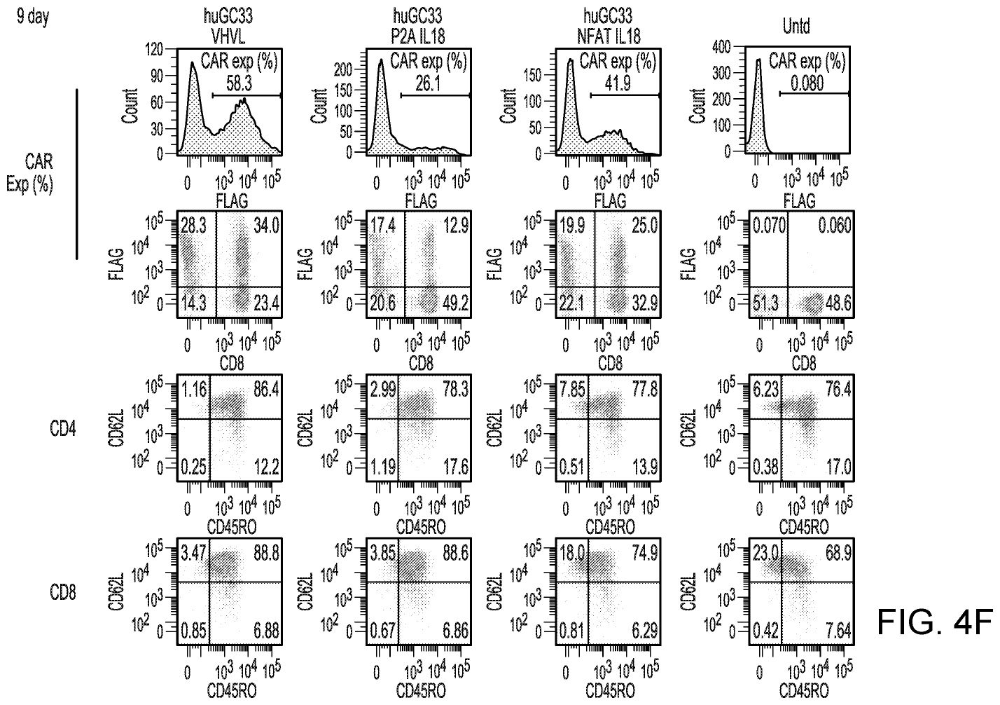

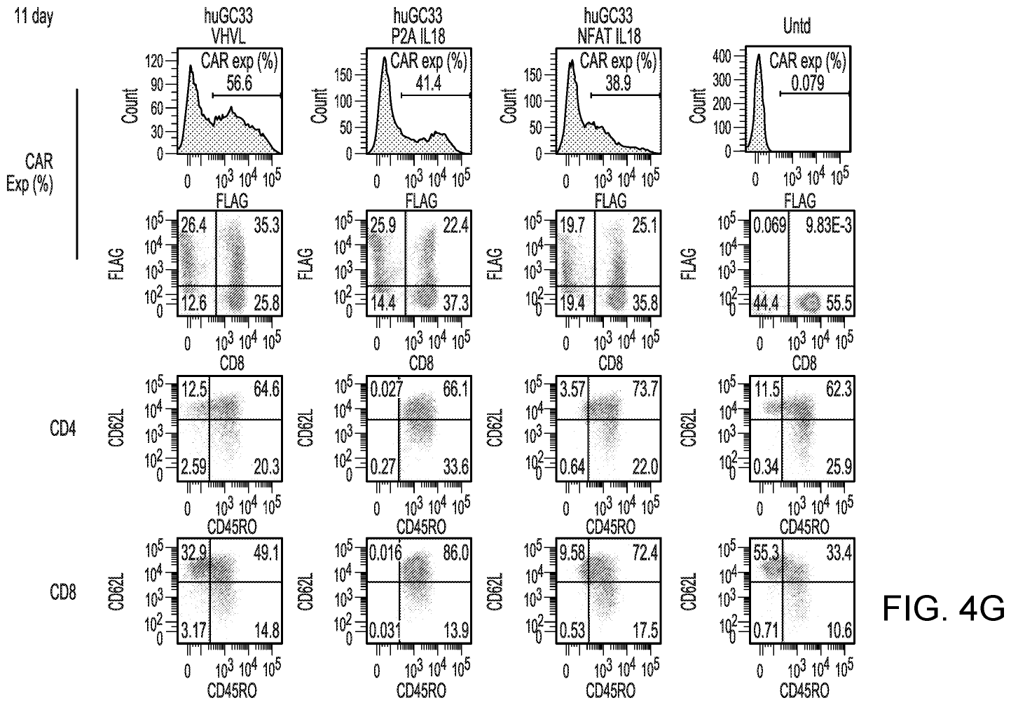

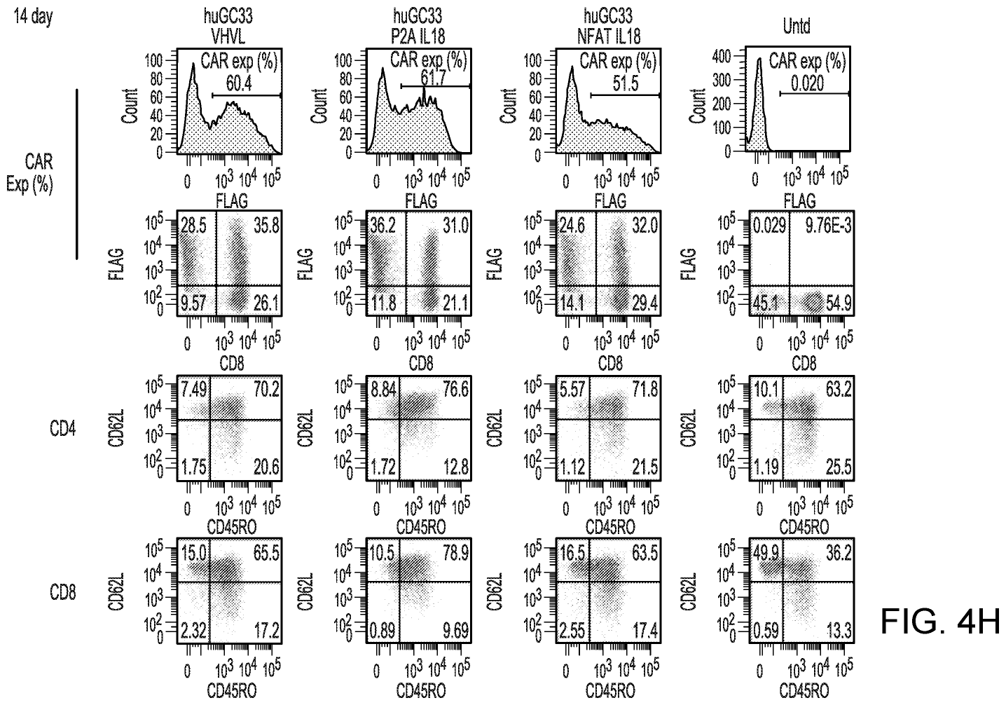

The huGC33-VHVL-P2A-IL18 and huGC33-VHVL-NFAT-IL18 CAR-T constructs were compared in vitro, wherein cell growth for each group of CAR-T cells on day 14 of cell culture was compared by total fold expansion (FIG. 4A). Cell expansion and cell viability from day 7 to day 14 were compared, and nearly all conditions demonstrated over 90% CAR-T cell viability, except huGC33-VHVL-P2A-IL18 CAR-T cells, which demonstrated cell viability of less than 90% on day 7, day 9, and day 11 (FIGS. 4B-4C). Luciferase based cytotoxicity was measured using a GPC3 positive cell line to harvest target cells while the CAR-T cells were used as effector cells. The cells were incubated at an E:T ratio of (effector (E):target (T))=10:1 in a 96-well white polystyrene microplate, then incubated with Bright-Glo™ Luciferase Assay reagent for 5 mins, and cytotoxicity was measured and quantified using a luminometer. Results show that in vitro killing activity was similar in all groups of CAR-T cells (FIG. 4D). Further, CAR expression was analyzed on day 7, day 9, day 11, and day 14 of cell culture using flow cytometry (FIGS. 4E-4H).

### Example 3—huGC33-VHVL-P2A-IL18 and huGC33-VHVL-NFAT-IL18 CAR-T Cells In Vivo

Cancer cells from human hepatocellular carcinoma cell lines, Huh-7-GL, PLC/PRF/5-GL (GPC3 positive cell lines) and SK-Hep-1 (GPC3 negative cell line) were injected into NSG mice (2×10⁶ cells/head) and the growth of the liver tumor was observed over a period of time. The animals were divided into groups according to the tumor size measured in each animal (Tables 7-10).

TABLE 7

| | | Injection | | |
|---|---|---|---|---|
| Groups | | materials | Route | # of mice |
| 1 | Non-Treated | 5% HSA | I.V Inj. | 5 |
| 2 | huGC33 VHVL | CAR-T | | |
| | 0.25 × 10⁶ cells | | | |
| 3 | huGC33 VHVL | | | |
| | 0.5 × 10⁶ cells | | | |
| 4 | huGC33 VHVL-P2A-IL18 | | | |
| | 0.25 × 10⁶ cells | | | |
| 5 | huGC33 VHVL-P2A-IL18 | | | |
| | 0.5 × 10⁶ cells | | | |
| 6 | huGC33 VHVL-NFAT-IL18 | | | |
| | 0.25 × 10⁶ cells | | | |
| 7 | huGC33 VHVL-NFAT-IL18 | | | |
| | 0.5 × 10⁶ cells | | | |

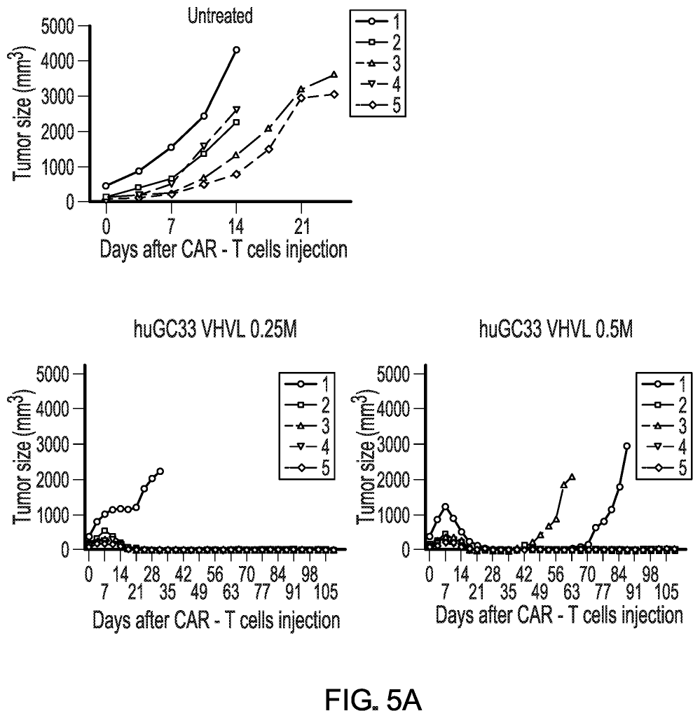

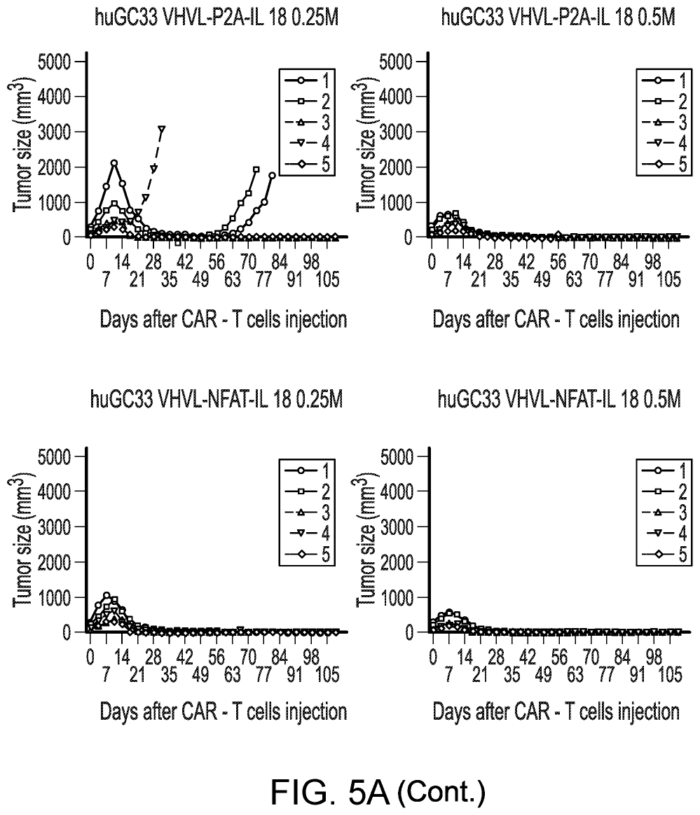

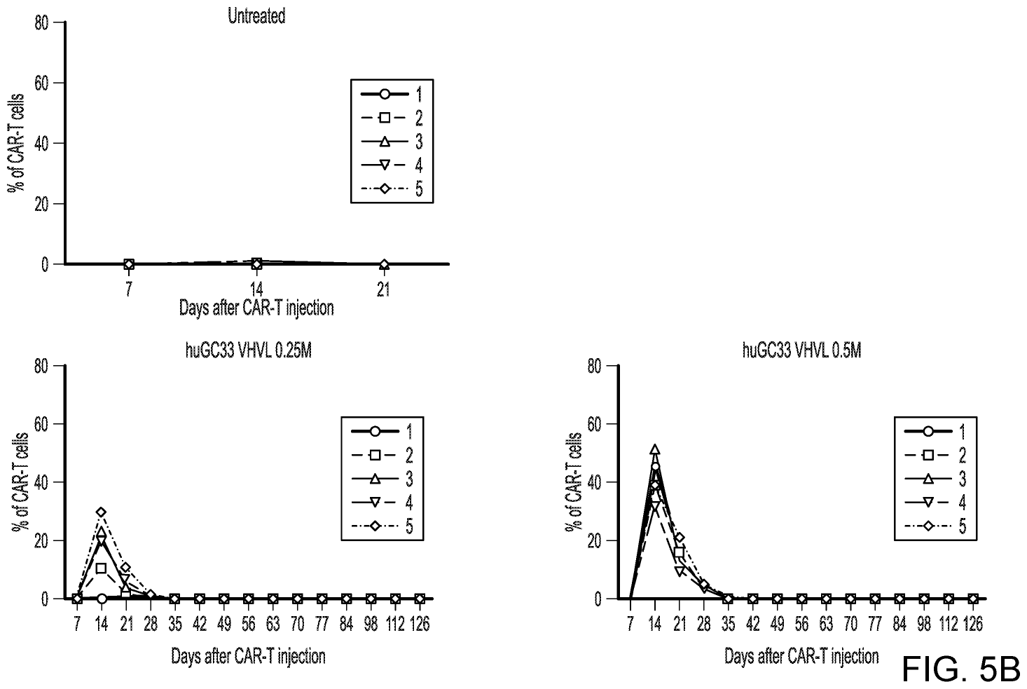

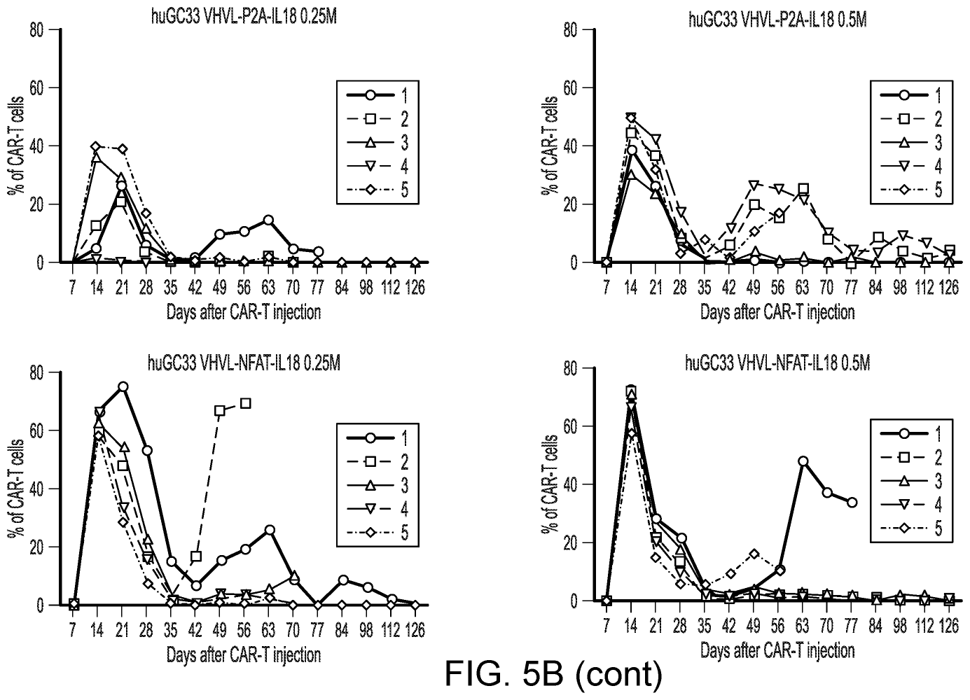

Huh-7-GL cells were injected into NSG mice (2×10⁶ cells/head) and after growth of the tumor, the huGC33-VHVL-P2A-IL18 CAR-T cells and huGC3-VHVL-NFAT-IL-18 CAR-T cells were injected. All mice in a control group which did not receive any injections (untreated group) died within 28 days after the time of injection, only one mouse from the huGC33 VHVL group died at week 5 after the injection, and three mice from the huGC33-VHVL-P2A-IL18 group died, while all mice in the groups which received injections of huGC33-VHVL-NFAT-IL18 CAR-T cells survived for at least 15 weeks after injection (FIG. 5A).

Observations for 15 weeks after GPC3 CAR-T cell injections showed that the group of mice which received injection of huGC33-VHVL-NFAT-IL18 showed the highest increase of level of CAR-T cells in the blood (FIG. 5B).

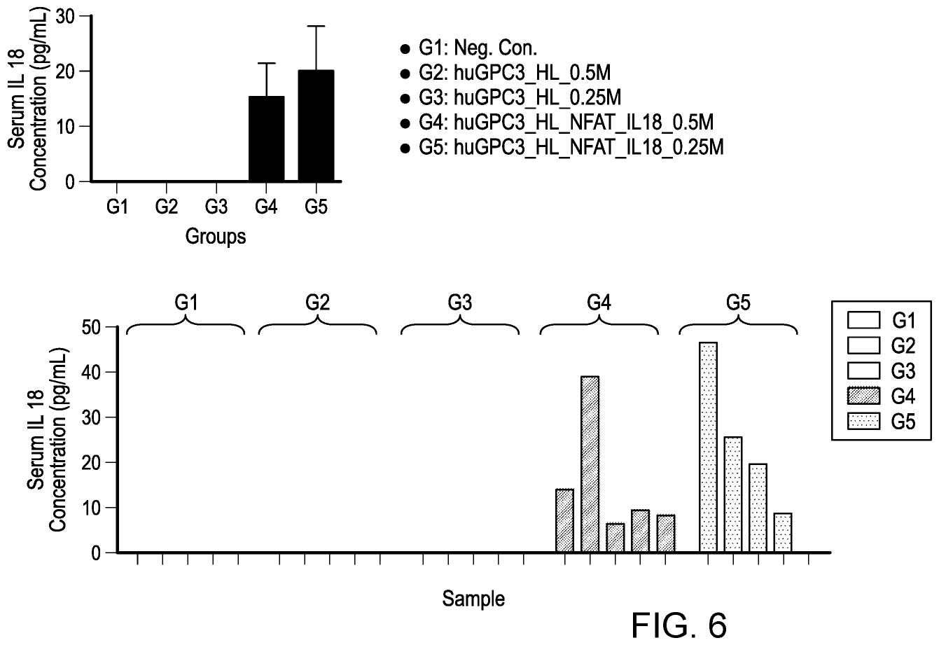

On day 14 after injections with the GPC3 CAR-T cells, blood serum from the mice was collected to analyze IL-18 within the blood serum using ELISA analysis. The results showed that the concentration of IL-18 in blood serum reached about 20 pg/mL only in the mice which received huGC33-VHVL-NFAT-IL18 CAR-T cell injections (FIG. 6).

### Example 4—huGC33-VHVL-NFAT-IL18 and huGC33-VHVL-IL18-NFAT In Vitro

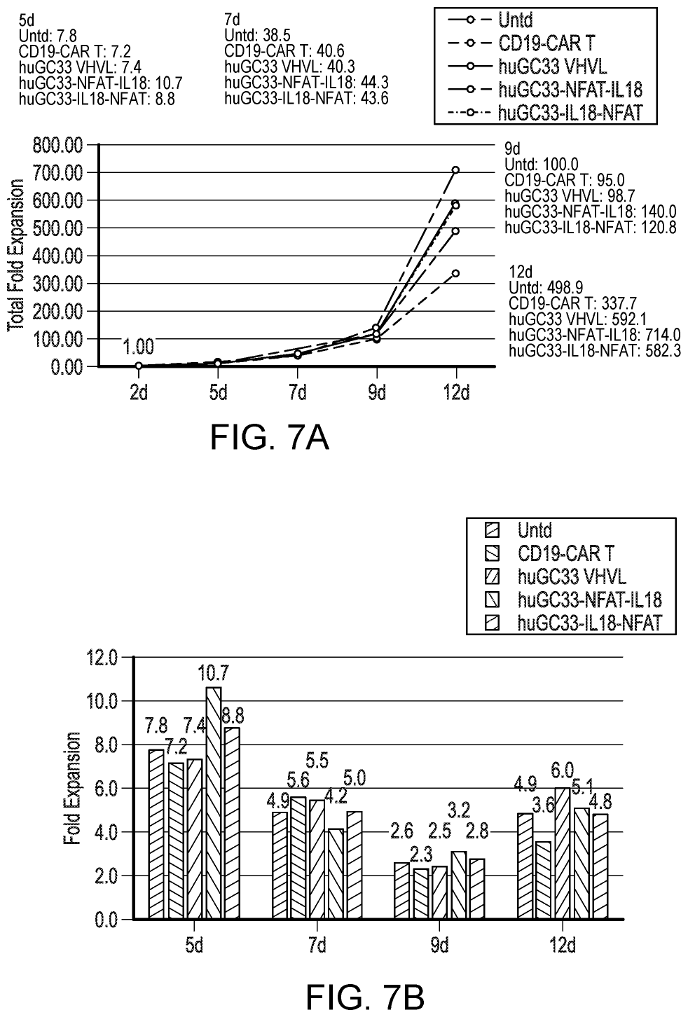

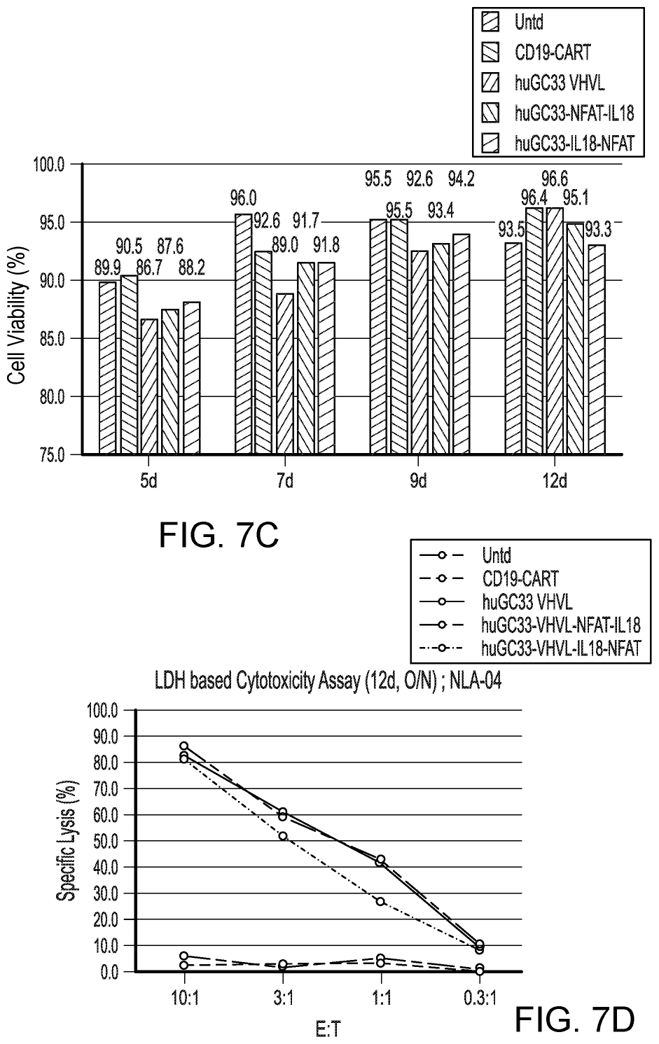

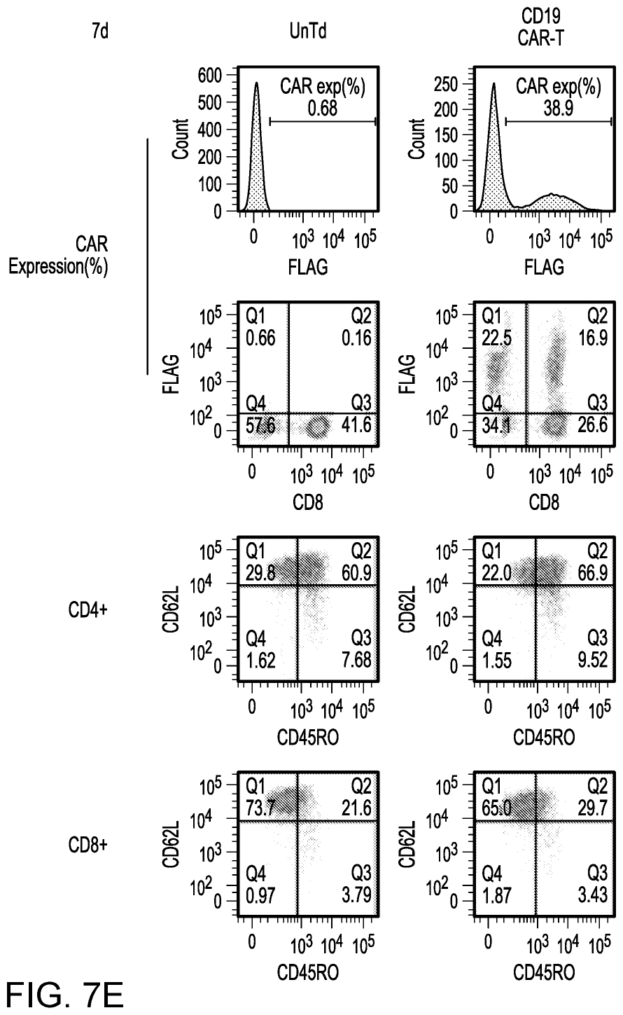

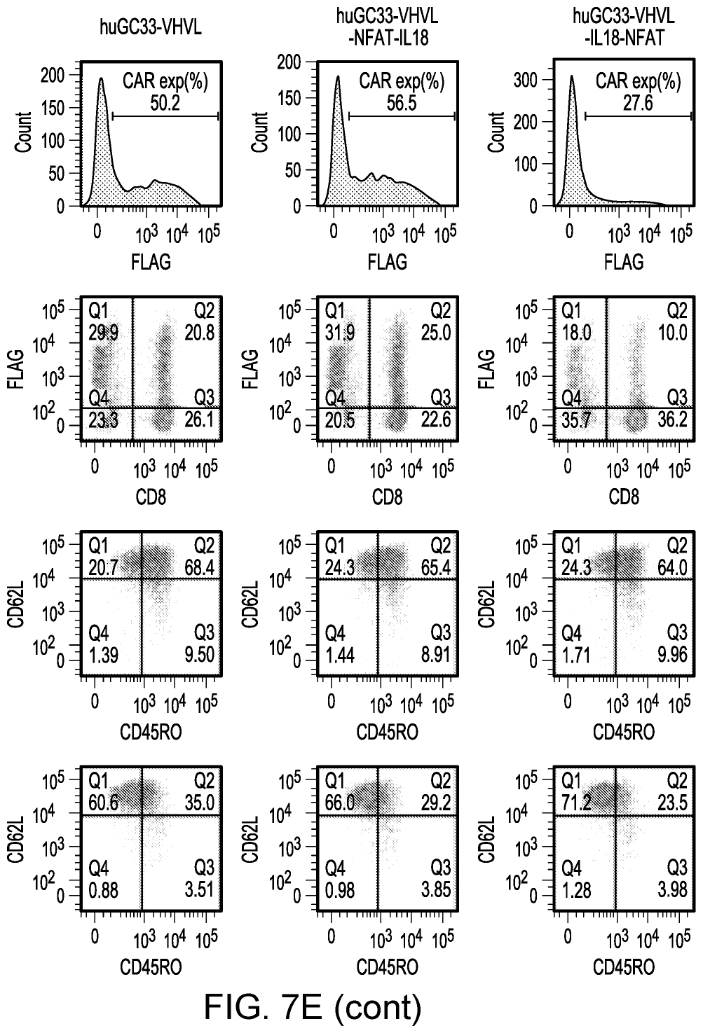

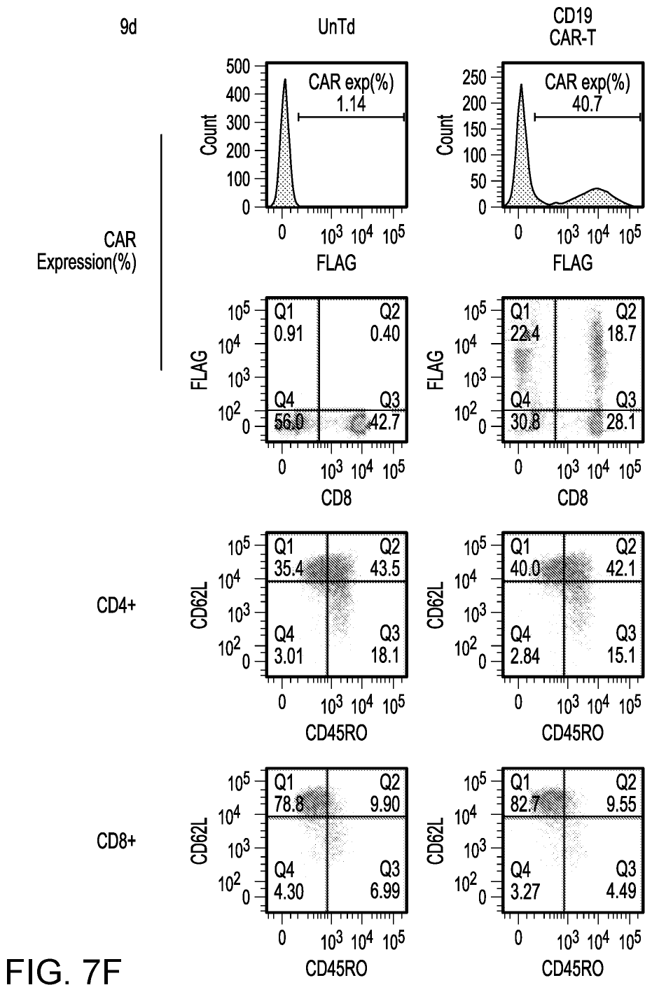

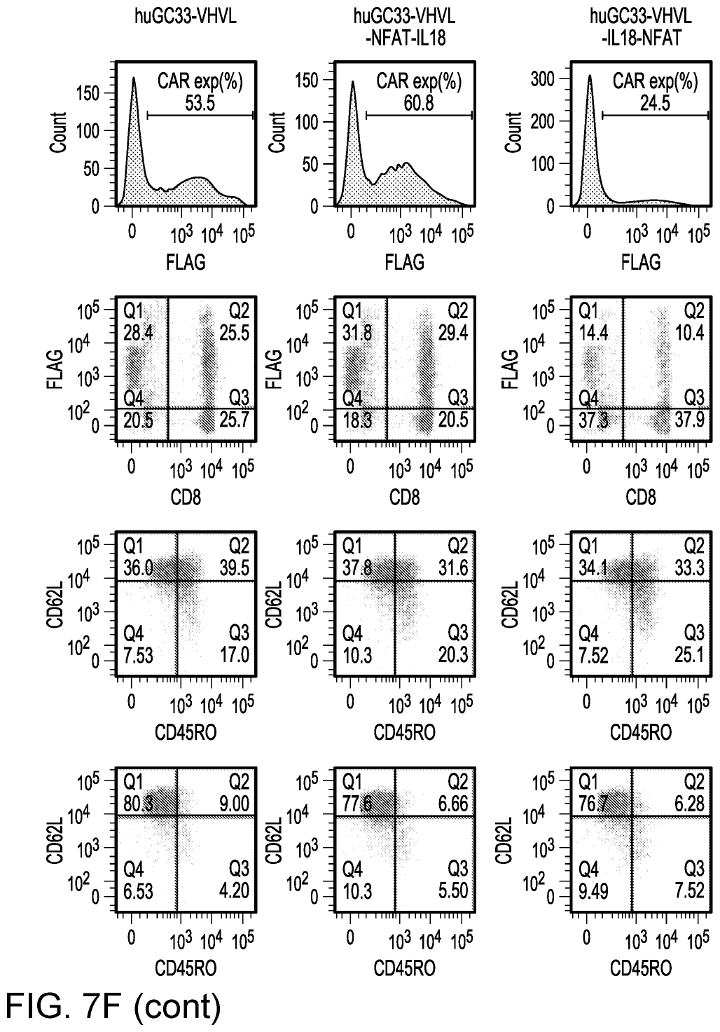

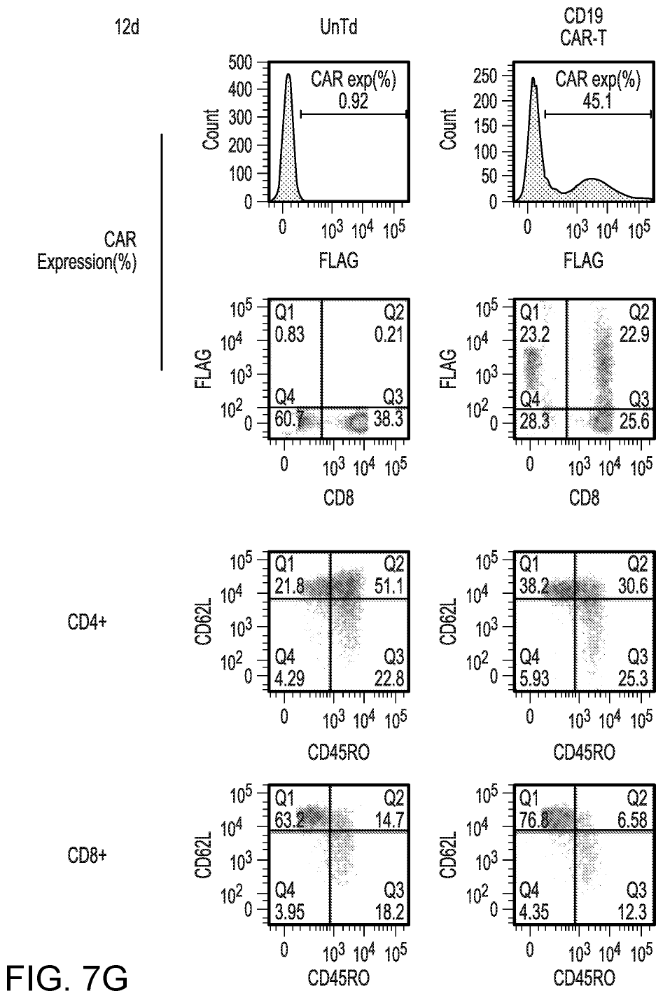

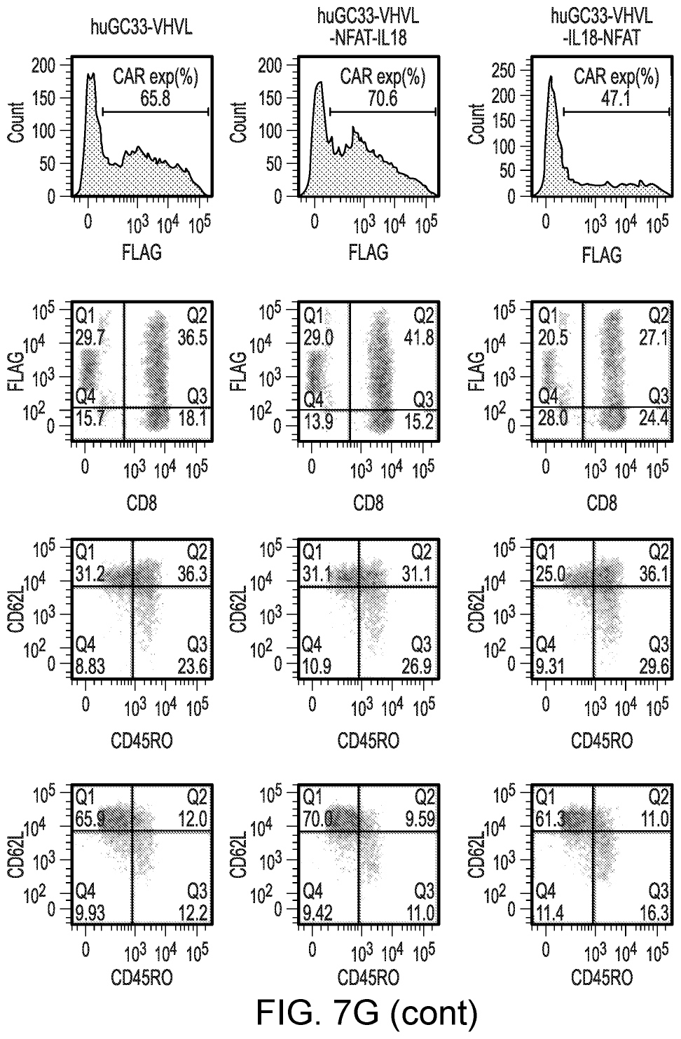

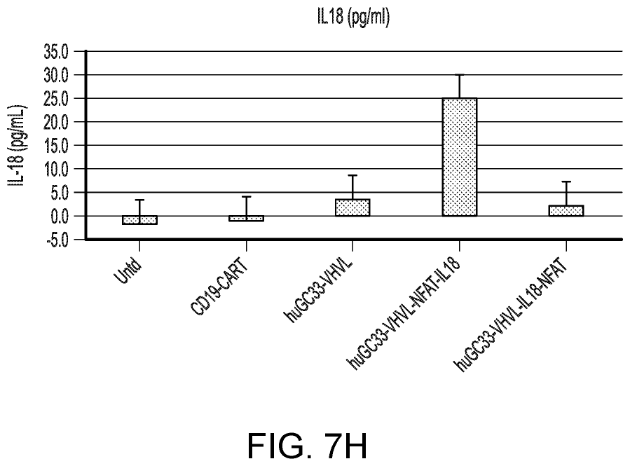

The huGC33-VHVL-NFAT-IL18 and huGC33-VHVL-IL18-NFAT CAR-T constructs were compared in vitro, wherein total fold expansion compares cell growth for each CAR-T cells on day 12 of cell culture (FIG. 7A). Cell expansion and cell viability from day 5 to day 12 were compared and shows over 90% viability for all CAR-T cells at 12 days (FIGS. 7B-7C). LDH-based cytotoxicity was measured using a GPC3 positive cell line to harvest target cells while the CAR-T cells were used as effector cells. The cells were incubated at a E:T ratio of (effector (E):target (T))=10:1 in a 96-well U bottom plate, then incubated with Cyto Tox96 reagent for 30 mins, and cytotoxicity was measured and quantified using a microplate reader at 490 nm wavelength (FIG. 7D) and CAR expression was analyzed using flow cytometry on day 7, 9, and 12 (FIGS. 7E-7G). Further, IL-18 concentration was analyzed using ELISA. Results show that huGC33 VHVL-NFAT-IL18 CAR-T cells showed highest IL-18 expression (FIG. 7H).

### Example 5—huGC33-VHVL-NFAT-IL18 and huGC33-VHVL-IL18-NFAT CAR-T Cells In Vivo

Huh-7-GL cells were injected into NSG mice (2×10⁶ cells/head) and after the size of the tumor reached either about 330 mm³ (Table 8) or about 1100 mm³ (Table 9) the mice were divided into groups which would receive no treatment, 0.25 million cells of CD19 CAR-T cells, 0.25 million cells of huGC33 VHVL, 0.1 million cells of huGC33 VHVL-NFAT-IL18, 0.25 million cells of huGC33 VHVL-NFAT-IL18, 0.1 million cells of huGC33 VHVL-IL18-NFAT, or 0.25 million cells of huGC33 VHVL-IL18-NFAT CAR-T cells. The huGC33-VHVL-NFAT-IL18 and huGC33-VHVL-IL18-NFAT CAR-T cells were then injected into each group of mice.

TABLE 8

| | | Injection | | # of mice |
|---|---|---|---|---|
| Groups | | materials | Route | per condition |
| 1 | Untreated | 5% HSA | I.V Inj. | 5 |
| 2 | CD19 | CAR-T | | |
| | 0.25 × 10⁶ cells | | | |
| 3 | huGC33 VHVL | | | |
| | 0.25 × 10⁶ cells | | | |
| 4 | huGC33 VHVL-NFAT-IL18 | | | |
| | 0.1 × 10⁶ cells | | | |
| 5 | huGC33 VHVL-NFAT-IL18 | | | |
| | 0.25 × 10⁶ cells | | | |
| 6 | huGC33 VHVL-IL18-NFAT | | | |
| | 0.1 × 10⁶ cells | | | |
| 7 | huGC33 VHVL-IL18-NFAT | | | |
| | 0.25 × 10⁶ cells | | | |

TABLE 9

| | | Injection | | # of mice |
|---|---|---|---|---|
| Groups | | materials | Route | per condition |
| 1 | Untreated | 5% HSA | I.V Inj. | 3 |
| 2 | huGC33 VHVL | CAR-T | | |
| | 0.25 × 10⁶ cells | | | |
| 3 | huGC33 VHVL-NFAT-IL18 | | | |
| | 0.1 × 10⁶ cells | | | |
| 4 | huGC33 VHVL-NFAT-IL18 | | | |
| | 0.25 × 10⁶ cells | | | |
| 5 | huGC33 VHVL-IL18-NFAT | | | |
| | 0.1 × 10⁶ cells | | | |
| 6 | huGC33 VHVL-IL18-NFAT | | | |
| | 0.25 × 10⁶ cells | | | |

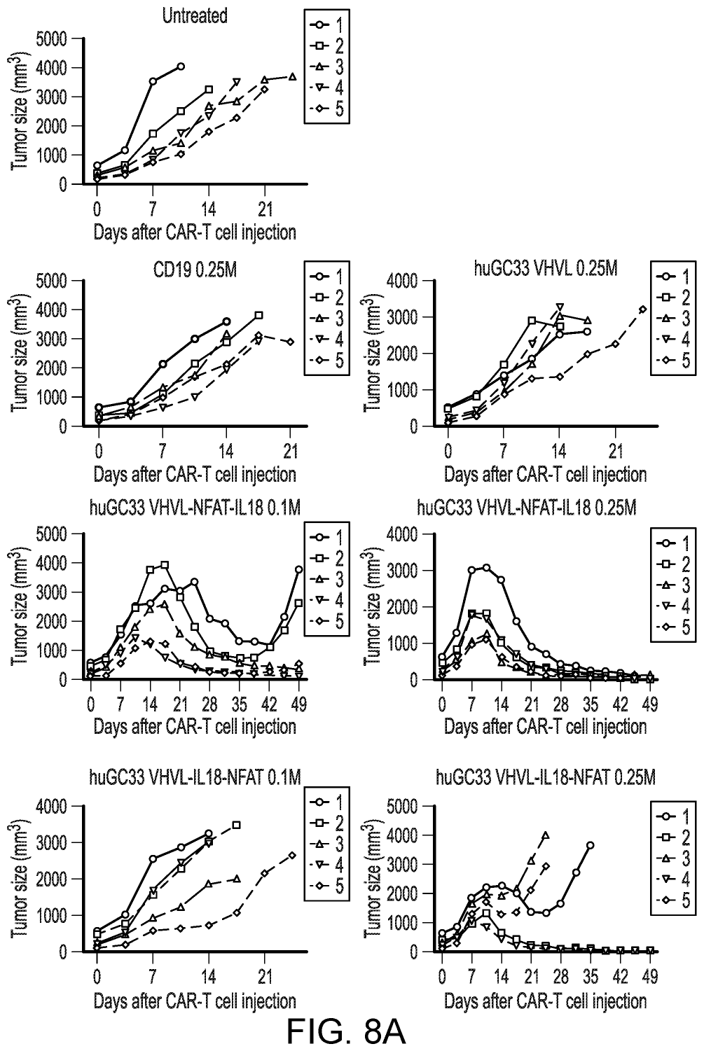

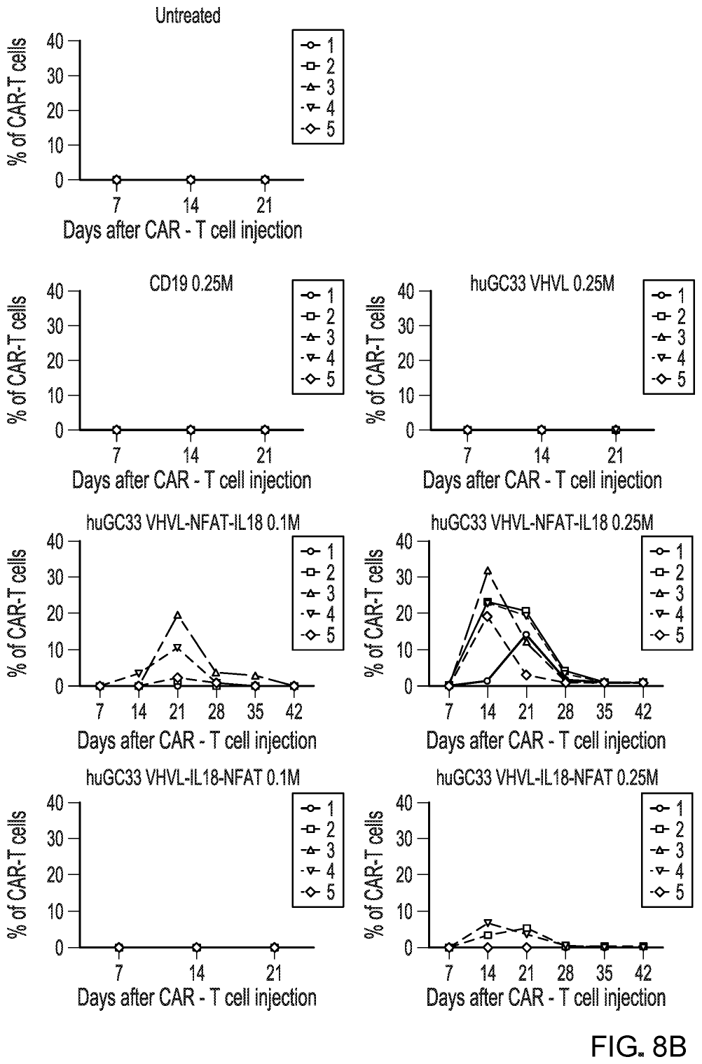

Results show that the group of mice for which the tumor size reached about 330 mm³, the untreated mice and mice injected with CD19 CAR-T cells all died within 24 days after injection. Only two mice of the group which received an injection of 0.1 million cells of huGC33-VHVL-NFAT-IL18 CAR-T cells died at day 49 after injection, while three mice in the group which received an injection of 0.25 million cells of huGC33-VHVL-IL18-NFAT CAR-T cells showed a slight decrease in tumor size but then died due to regrowth of the tumor. Results from the group which received an injection of 0.1 million of huGC33-VHVL-IL18-NFAT CAR-T cells showed that the injection did not show any effectiveness of reducing tumor size. (FIG. 8A). Observations for 6 weeks after GPC3 CAR-T cell injections showed that only mice with reduced tumor size showed an increase of level of CAR-T cells in the blood followed by a decrease after tumor size was reduced (FIG. 8B).

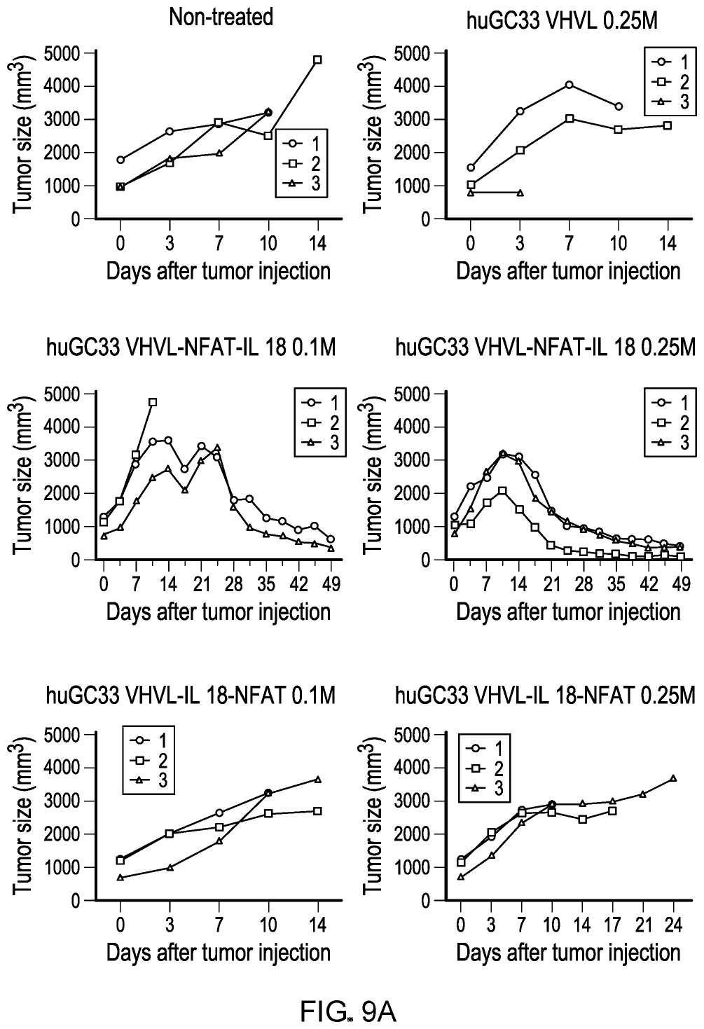

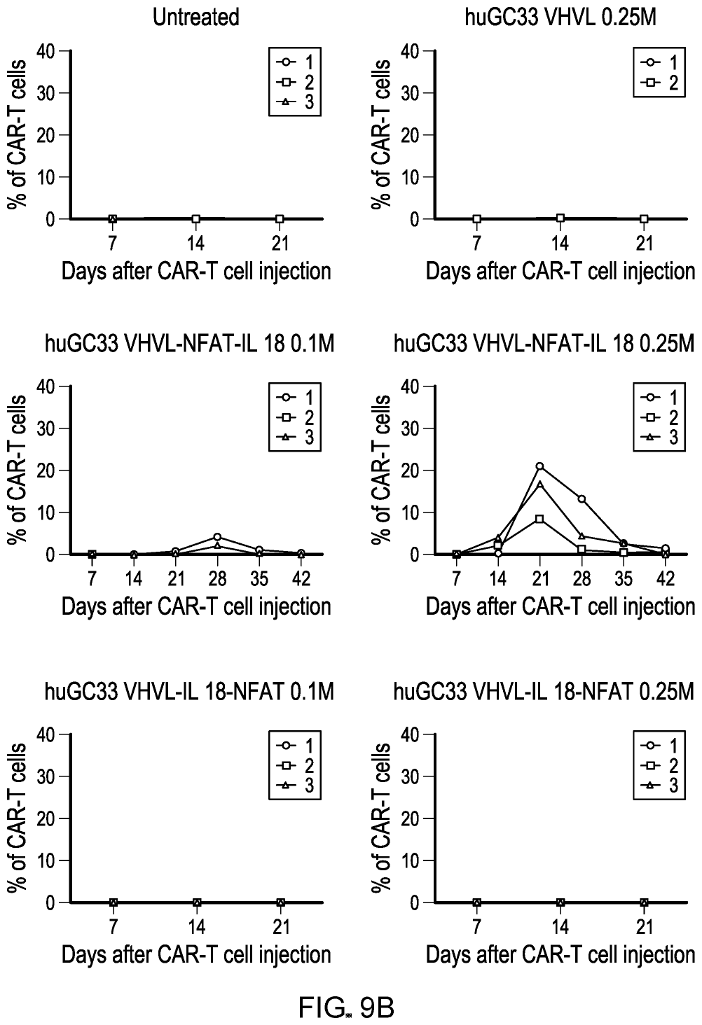

For the group of mice for which the tumor size reached about 1100 mm³, only mice with huGC33-VHVL-NFAT-IL18 showed reduction in tumor size until day 49 after injection while all mice in other groups died within 24 days after injection (FIG. 9A). CAR-T cell levels in the blood were shown to be slightly lower than that of the group of mice for which the tumor size reached about 330 mm³ (FIG. 9B).

### Example 6—huGC33 VHVL-NFAT-IL18 In Vivo with PLC/PRF/5-GL and SK-Hep-1 Cell Lines

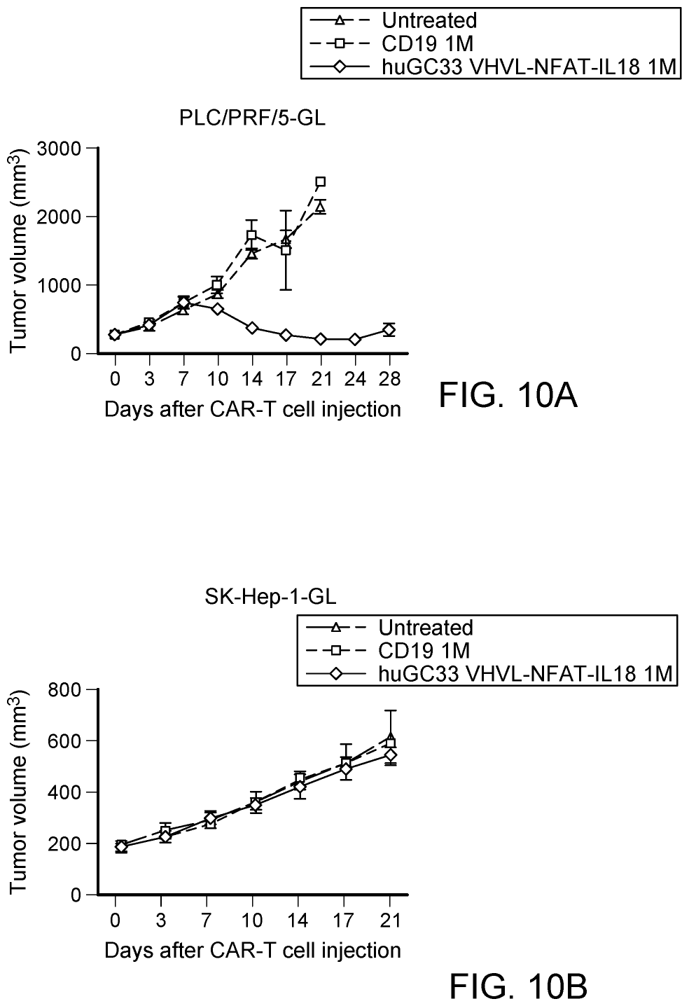

Cancer cells from PLC/PRF/5-GL cell line which shows low GPC3 expression were injected into NSG mice (2×10⁶ cells/head) and the growth of liver tumor was observed over a period of time. Results show that the group which received injections of huGC33 VHVL-NFAT-IL18 showed a decrease in tumor size compared to all mice in the untreated and CD19 groups, which died within 21 days after the time of injection, thereby indicating that huGC33 VHVL-NFAT-IL18 CAR T cells effectively reduced tumor size in GPC3 positive cancer cells (FIG. 10A).

When huGC33 VHVL-NFAT-IL18 CAR T cells were injected into SK-Hep-1-GL cancer cells, a GPC3 negative cell line, no mice demonstrated any reduction in tumor size and all mice died within 21 days after time of injection. This demonstrates that the GPC3 CARs expressing IL-18 used here are specific for GPC3 expressing tumor cells. (FIG. 10B).

TABLE 10

| | | Injection | | |
|---|---|---|---|---|
| Group | | materials | Route | # of mice |
| 1 | Untreated | 5% HSA | I.V Inj. | 5 |
| 2 | CD19 | CAR-T | | |
| | 1 × 10⁶ cells | (5% HSA) | | |
| 3 | huGC33 VHVL-NFAT-IL18 | | | |
| | 1 × 10⁶ cells | | | |

These results show that huGC33-VHVL-NFAT-IL18 CAR-T cells are effective in maintaining an effective concentration of CAR-T cells in blood serum while showing higher survival ratings.

1. An immune cell comprising:
a chimeric antigen receptor (CAR), wherein the CAR comprises an extracellular antigen-binding domain that binds specifically to glypican-3 (GPC3), wherein the extracellular antigen-binding domain comprises:
a light chain variable domain comprising VL CDRs 1, 2, and 3 and a heavy chain variable domain comprising VH CDRs 1, 2, and 3, wherein
a. the VL CDRs 1, 2, and 3 comprise SEQ ID NOs: 1, 2, and 3, and
b. the VH CDRs 1, 2, and 3 comprise SEQ ID NOs: 4, 5, and 6, and
a transmembrane domain, and an intracellular signaling domain; and
an exogenous nucleic acid comprising a sequence encoding interleukin-18.

2. The immune cell of claim 1, wherein the light chain variable domain comprises a sequence that is at least 80% identical to SEQ ID NO: 10.

3. (canceled)

4. (canceled)

5. The immune cell of claim 1, wherein the heavy chain variable domain comprises a sequence that is at least 80% identical to SEQ ID NO: 8.

6. (canceled)

7. (canceled)

8. The immune cell of claim 1, wherein the interleukin-18 is a human interleukin-18.

9. The immune cell of claim 8, wherein the human interleukin-18 comprises a sequence that is at least 80% identical to SEQ ID NO: 11 or 12.

10. (canceled)

11. (canceled)

12. The immune cell of claim 1, wherein the sequence encoding interleukin-18 further includes a sequence encoding a secretion signal sequence.

13. The immune cell of claim 12, wherein the secretion signal sequence is an interleukin-2 secretion signal sequence.

14. The immune cell of claim 13, wherein the interleukin-2 secretion signal sequence comprises a sequence of SEQ ID NO: 13 or 14.

15. The immune cell of claim 1, wherein the exogenous nucleic acid further comprises a promoter operably linked to the sequence encoding interleukin-18.

16. The immune cell of claim 15, wherein the promoter is a constitutive promoter.

17. The immune cell of claim 15, wherein the promoter is an inducible promoter.

18. The immune cell of claim 15, wherein the promoter is an NFAT promoter.

19. The immune cell of claim 1, wherein the antigen-binding domain is humanized.

20. The immune cell of claim 1, wherein the antigen-binding domain is human.

21. The immune cell of claim 1, wherein the antigen-binding domain is an scFv.

22. The immune cell of claim 1, wherein the transmembrane domain is a transmembrane domain selected from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11 b, CD11c, CD11 d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6.

23. (canceled)

24. The immune cell of claim 1, wherein the intracellular signaling domain comprises an intracellular signaling domain from a protein selected from the group consisting of: 4-1BB/CD137, an activating NK cell receptor, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3delta, CD3epsilon, CD3gamma, CD3zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRTAM, a cytokine receptor, DAP-10, DNAM1 (CD226), Fc gamma receptor, GADS, GITR, HVEM (LIGHTR), IA4, ICAM-1, Ig alpha (CD79a), IL-2Rbeta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08, lymphocyte function-associated antigen-1 (LFA-1), a MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecules (SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6, or any combination thereof.

25. (canceled)

26. (canceled)

27. (canceled)

28. (canceled)

29. (canceled)

30. (canceled)

31. The immune cell of claim 1, which secretes the IL-18 encoded by the exogenous nucleic acid.

32. (canceled)

33. (canceled)

34. A method of treating a subject having a glypican-3-associated cancer, the method comprising administering to the subject an immune cell of claim 1.


SEQUENCE LISTING (22 sequences)

SEQ ID NO: 1 - protein, 16 aa, Artificial
  Light chain CDR1
RSSQSLVHSNGNTYLH

SEQ ID NO: 2 - protein, 7 aa, Artificial
  Light chain CDR2
KVSNRFS

SEQ ID NO: 3 - protein, 9 aa, Artificial
  Light chain CDR3
SQNTHVPPT

SEQ ID NO: 4 - protein, 5 aa, Artificial
  Heavy chain CDR1
DYEMH

SEQ ID NO: 5 - protein, 17 aa, Artificial
  Heavy chain CDR2
ALDPKTGDTAYSQKFKG

SEQ ID NO: 6 - protein, 6 aa, Artificial
  Heavy chain CDR3
FYSYTY

SEQ ID NO: 7 - DNA, 345 bp, Artificial
  Heavy chain variable region
caagtgcaactcgtacaatcaggtgctgaagtcaaaaagccgggagcctctgttaaagtgtcctgtaaagccagcggctacacctttaccgattatgagatgcactgggttcggcaggctccgggccaaggtctcgagtggatcggggctcttgacccaaagacgggcgacacggcttattcacaaaaattcaaaggtagggctactctgactgccgataagtccaccagcaccgcgtatatggagctctctagcttgcgaagcgaggacacggcggtgtactattgcacacgcttctatagttacacatattggggtcaaggcacgcttgtgaccgtgtctagc

SEQ ID NO: 8 - protein, 115 aa, Artificial
  Heavy chain variable region
QVQLVQSGAEVKKPGASVKVSCKASGYTFTDYEMHWVRQAPGQGLEWIGALDPKTGDTAYSQKFKGRATLTADKSTSTAYMELSSLRSEDTAVYYCTRFYSYTYWGQGTLVTVSS

SEQ ID NO: 9 - DNA, 336 bp, Artificial
  Light chain variable region
gacgtcgttatgacacagagtcccctctccttgccggtgaccctgggtcagcctgcgtccatctcttgcagatcctcccagtctctggtacactccaacggcaacacatacttgcactggtaccaacaaagacctggtcagtcaccgcgacttctcatatataaagtttccaataggttcagtggagtgccagacaggttcagtggttcaggatcaggcactgatttcacgcttaaaatcagtcgggttgaggcggaggacgtaggagtttactattgcagccagaatacgcacgtgccgcctacttttggctctggaaccaagttggaaataaag

SEQ ID NO: 10 - protein, 112 aa, Artificial
  Light chain variable region
DVVMTQSPLSLPVTLGQPASISCRSSQSLVHSNGNTYLHWYQQRPGQSPRLLIYKVSNRFSGVPDRFSGSGSGTDFTLKISRVEAEDVGVYYCSQNTHVPPTFGSGTKLEIK

SEQ ID NO: 11 - DNA, 474 bp, Homo sapiens
tactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 12 - DNA, 474 bp, Homo sapiens
ctagtcttcgttttgaacagtgaacattatagatctatcccccaattcatcctcttttttcaaaatgagtttaaaaaggtctctctctttttcacaagctagaaagtatccttcgtatgatgaagattcaaattgcatcttattatcatgtcctgggacacttctctgaaagaatatgatgtcactttttgtatccttgatgttatcaggaggattcatttccttaaaggaaataattttgttctcacaggagagagttgaaattttctcacacttcacagagatagttacagccatacctctaggctggctatctttatacatacttataataaatatggtccggggtgcattatctctacagtcagaatcagtcatatcttcaaatagaggccgatttccttggtcaatgaagagaacttggtcattcaaatttcttatgactgataatttagattcaagcttgccaaagta

SEQ ID NO: 13 - DNA, 60 bp, Homo sapiens
atgtacaggatgcaactcctgtcttgcattgcactaagtcttgcacttgtcacaaacagt

SEQ ID NO: 14 - DNA, 60 bp, Homo sapiens
actgtttgtgacaagtgcaagacttagtgcaatgcaagacaggagttgcatcctgtacat

SEQ ID NO: 15 - DNA, 726 bp, Artificial
  huGC33 VHVL
caagtgcaactcgtacaatcaggtgctgaagtcaaaaagccgggagcctctgttaaagtgtcctgtaaagccagcggctacacctttaccgattatgagatgcactgggttcggcaggctccgggccaaggtctggagtggatcggggctcttgacccaaagacgggcgacacggcttattcacaaaaattcaaaggtagggctactctgactgccgataagtccaccagcaccgcgtatatggagctctctagcttgcgaagcgaggacacggcggtgtactattgcacacgcttctatagttacacatattggggtcaaggcacgcttgtgaccgtgtctagcggtggcggcggaagtggtggtggtggttctgggggcgggggttccgacgtcgttatgacacagagtcccctctccttgccggtgaccctgggtcagcctgcgtccatctcttgcagatcctcccagtctctggtacactccaacggcaacacatacttgcactggtaccaacaaagacctggtcagtcaccgcgacttctcatatataaagtttccaataggttcagtggagtgccagacaggttcagtggttcaggatcaggcactgatttcacgcttaaaatcagtcgggttgaggcggaggacgtaggagtttactattgcagccagaatacgcacgtgccgcctacttttggctctggaaccaagttggaaataaag

SEQ ID NO: 16 - DNA, 15 bp, Artificial
  additional 5 amino acids
cgtttctctgttgtt

SEQ ID NO: 17 - DNA, 126 bp, Artificial
  4-1BB intracellular domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 18 - DNA, 141 bp, Artificial
  4-1BB intracellular domain with five additional amino acids (euBBz)
cgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - DNA, 339 bp, Artificial
  CD3-zeta
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 20 - DNA, 66 bp, Artificial
  P2A
ggcagcggcgccacaaacttctctctgctaaagcaagcaggtgatgttgaagaaaaccccgggcct

SEQ ID NO: 21 - DNA, 301 bp, Artificial
  NFAT
acgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctcctcgaggacattttgacacccccataatatttttccagaattaacagtataaattgcatctcttgttcaagagttccctatcactctctttaatcactactcacagtaacctcaactcctgc

SEQ ID NO: 22 - DNA, 237 bp, Artificial
  poly A
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatc